(19) United States
(12) Patent Application Publication
(10) Pub. No.: US 2004/0120844 A1
(43) Pub. Date: Jun. 24, 2004

(54) Advanced oxidation of dangerous chemical and biological sources

(76) Inventors: Zamir Tribelsky, Mevaseret-Tzion (IL); Michael Ende, Moshav Shoeva (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/433,776

(22) PCT Filed: Dec. 7, 2001
(86) PCT No.: PCT/IL01/01137

(30) Foreign Application Priority Data
Dec. 7, 2000 (IL) 140180

(51) Int. Cl. A01N 1/00; A61L 2/00; C23F 11/00

(52) U.S. Cl. 422/2; 422/1; 422/22; 422/38

(57) ABSTRACT

Advanced Oxidation Technologies (AOT) using laser triggered and driven AOT platform are disclosed, including a method for the advanced oxidation of dangerous chemical and biological sources suspected in particular regions, a variety of uses of said method, and the environments where it can be implemented.

The method has two basic steps that are;

(c) spraying the regions to be treated with a cloud of gas, vapors, microdroplets, droplets, or bubbles formed from at least one liquid solution containing at least one type of photocatalitic oxidizing substance;

(d) directing across said cloud at least one high intensity beam of light having wavelength of between 220 and 390 nanometer for triggering said cloud thereby causing a catalyzed activation that releases free radicals of said oxidizing substance in order to react with said chemical or biological sources.

Various types and embodiments of systems and devices using the method of the present invention are also disclosed, including a bubble generator adapted for implementation of the method in various sites where treatment procedures according to the method are required.

### FIELD OF THE INVENTION

The present invention relates to Advanced Oxidation Technologies (AOT) using laser triggered and driven AOT platform.

More specifically, the present invention relates to a method for the advanced oxidation of dangerous chemical and biological sources suspected in particular regions. The present invention further relates to the various uses of said method, and environments where it is useful.

The present invention further relates to various embodiments of systems and devices using the method of the present invention, and to a bubble generator useful and adapted for implementation of the method in various sites where treatment procedures according to the method are required.

### BACKGROUND OF THE INVENTION

Scientist, engineers, bio-technologists, and producers, and end users have been searching for years for a non invasive disinfection technology, such technology which does not require any physical contact with the medium to be treated, and thus may provide a solution in manufacturing sites, mass production of bottled mineral waters, and food, drink, and beverages industries around the world. Further more, the type of “non invasive treatment technologies available today include: Gamma rays, X rays, Y rays, photons, radio waves, micro-waves, and several types of ionizing radiation. These available non invasive instrumentation are often dangerous, expensive, and require substantial periodical maintenance and replacements. Furthermore, instrumentation using such ionizing radiation types, require sophisticated support means, and infrastructure safety measurements, further complicating design criteria, and implementation. Several of these radiation types have already confirmed as causing cancer, and public confidence in these technologies at manufacturing plants is declining. Stringent legislation, and standards further fuel the need for alternative, safer, more economical methodologies for non interfering treatment. Conventional chemical technologies are limited since there is always the need to clear the liquids, and gases (of the “harmful” chemicals), and remove them from the specific volume to be consumed (i.e. after disinfection, or purification have occurred) once they have finished their useful cycle, or their disinfection, and oxidation activities. Recent activities in solid state electronics, lasers, and advanced polymer for optronics, laser pumping architectures, all have contributed to production of light sources small enough to be incorporated, or integrated (FIG. 1, 1-30) into system in close proximities to bottles, lids, corks, packaging production processes, and wide variety of packaging for foods, drinks, and many biotechnology products, and applications. Evolution of waveguide technology, and polymer production techniques making packages from material selected from at least one PET packaging layer/s, or polyolefin, or polyamide, or polycarbonate, or polyesteramide, or polyester or any resine combination thereof having refractive index profile for partial, or total internal reflection therein for equalizing the concentration of bacteria, viruses, cysts, phatogens, or biological, or organic, non organic, or toxic or noxious species therein. All have further contributed to much higher levels of biocompatibility, and interconnectivity, and interoperability to commercial mass production environments.

As introduced in the field of the invention, the subject of the present invention includes environmental protection, and the protection of public health, and tools associated with domestic industrial, medical, engineering and environmental fields, using pulsed power technology in the UVA/B/C. The driving principle behind the technology is synchronized control of the time domain in which appropriate dose of light is delivered into specified geometry, or predetermined surface area causing specific electro-optical effects. More specifically, the present invention discloses a novel methodology for surface disinfection, and purification, and for dimensional treatment platform, by facilitating the catalytic formation of radical species (such as OH*) barrier technology (CFRS), a layer which upon triggering turns to “fire wall” (thus preventing passage through of noxious species due to the short duration of time [Fs] in which the catalytic processes occurs) made out of multiple layers of highly radical species, lasting extremely short fraction of time, for the purpose of forming photoreactive layer wherein the preferred mode for advance Catalytic Oxidation, Electro catalytic Oxidation, photolysis, and photo dissociation (of upper surface layers of medical instrumentation, as well as actual damaged tissues of the Human body) occurs.

More specifically, the present invention facilitate the provision of a protective photoreactive barrier technology (PPRBT), by means and utilization of light, liquid, gasses, and optronic time domain triggering (such as can be produced by high peak power high rep, rate uv lasers) the present invention is beneficial in cases wherein external cuts may hinder physiological activity, and may lead for infection, or further complications. Further more, the present invention facilitate phototreatment of surfaces faster then any previous methodology in the field by using photocatalysis, electrocatalysis, and hybridazation techniques. The novel methodology of he present invention facilitate the protection of large areas from harmful effects of contact with bacteria, or from contact with noxious or poisonous species.

By calibrating the action spectrum (i.e. absorption, transmitance, transparency, refractive index, or refractive index profile of the air/body, air/instrument, liquid or gas encapsulating layer (i.e. the layer “barrier between the surface of the actual instrumentation or body surface, and enterinmg/triggering beams of pulsed light, and the required coupling of light to the surface to be treated, thus taking into account specie specific wavelength interactions of the laser light (source used in accordance with the methodology of the present invention), the present invention offer economical solution using energy efficient methodology, and extremely safe operation procedures, requiring no skill operators, or special complex hardware procedure. The method of the present invention is simple to implement, and include fully automatic procedure management, so easy integration intro already made set up is simplified, and conserve time and energy during integration, installation, and operation procedures.

More specifically, the method of the present invention by using for example, a pulsed, high repetition rate, high peak power laser light sources, facilitate the formation of high energy density zone through which liquids, or gasses carrying contamination, or may be penetrated by invading antigens, are thus treated according to the methodology of the present invention.

One best mode among the many utilizing the method of the present invention is especially beneficial for disinfection of wide variety of medical instrumentation.

Further more, the methodology disclosed by the present invention offer solution for medical procedures requiring short working cycles, and thus offer important benefits in terms of shorter duty cycles, faster processing time, safer inactivation/Dissociation effectiveness, and important capital savings due to the novelty of the methodology of the present invention for photocatalytic protection of medical instrumentation or surfaces, or dimensions, or volumes by using UVA, UVB, UVC light produces by high repetition rates, high peak power lasers.

Modern society encapsulating a wide diversity of cultures, and professional occupations has often created population unrest, exposing challenges to leaders in search of peace, prosperity and positively charged commercial interactions.

Certain factors effecting the depletion, and contamination of natural water AND air resources, population expansion, global warming, modern, competitive world of man often riddle with socioeconomic positioning, conflicts in need of resolutions is presenting challenges associated with a diversity of eventualities requiring proficient, and capable technologies. Scientific, engineering, and industrial producers, and end users are engaged intensively in the search for new innovative technologies capable of offering solutions. Several evolutionary steps towards technologies that surpass limitations imposed by currently used chemical methodologies, and backed by trends and progress in such fields as solid state electronics, electro-optic fabrication techniques, photo-chemistry, photo-catalysis, and photo-electro-catalysis and laser technologies. When combined expertise in these fields are extrapolated against the geographical locations factor for different nations of the world, and population concentration driven by interests of accessibility to natural resources (such as water and air), and in the context of often-destructive and sinister nature of certain industries engage in the preparation of noxious species, from biological and/or chemical origins. Awareness is growing rapidly, that such noxious species may be used against fellow man illegally.

In view of the above scientists, engineers, technologists, producers, and end users have all been searching for the perfect technology able to offer adequate protection of bio-terrorism, and terrorist attacks using unconventional weapons.

The present invention disclosed a novel methodology wherein the destructive effects of such threatening eventualities are minimized, and or eliminated in situ, in real time and for wide variety of terrain and applications. More specifically, the methodology of the present invention discloses method and related devices for the protection of mankind, animal and live stock, key building installation, metropolitan areas, and large surface, volume or dimension areas in almost any geometrical utilization required for specific applications. The methodology of the present invention challenge strict limitation imposed by use of current chemical, radiation means, or temperature sterilization techniques. Current methodologies use mainly chemical disinfectant (applying, spraying, or distributing) formulated with toxic compounds capable of damaging the treated area as well as the noxious species within it. A single perfect chemical disinfectant for all eventualities of contamination (or cross contamination) does not exist, as it will have to poses so many features at once, however, even in the event that a multi components suspension, and complex chemical compounding volumes will in most cases facilitate the formation of toxic residual effects, DBPs (Disinfection By Products). Currently used chemical agents (or reagents) accumulate, and can penetrate the surface of the ground, reaching natural water resources, and under-ground aquifers, when above ground, these compounds may endanger living beings, animal and plants. More specifically, given the chemical toxic nature, or biological noxious threatening aspects of these compounds, associated relevant transport, storage and application delivery aspects are often cumbersome, expensive, and require highly skilled human resources and infrastructure for safe, prolonged, and repeatable industrial operation. Effective operability, interconnectivity, and interoperability is thus difficult to achieve with currently used technologies. Radiation chemistry has already proven that Gamma rays, electron beams, and conventional mercury based UV lamps fail to provide adequate protection against the diversity of geometries involved, and for reason of feasibility and practicality. Currently used UV lamps (medium pressure, low pressure, LPHO (Low Pressure High Output) cannot provide efficient disinfection, sterilization or dissociation capabilities due to their polychromatic spectral nature. More specifically, mercury (the principle means for generating UV from lamps is already recognized as extremely toxic substance, and it is a self limited technology. More specifically, the more pressure a UV lamp contain, the higher the energies that could be generated from the lamp, however the actual energy the lamp will produce will almost not include any UV (more pressure=more power, but no UV at output), lower pressure will yield less power, but much more relevant UV at output.

In contradistinction to conventional, currently used technologies in the field, the non chemical, non toxic methodology of the present invention is not so limited, that why the method of the present invention and devices using same, could be used for wide variety of applications in numerous fields offering open architecture modular design platform for maximum adaptability and rapid response to threatening eventualities involving liquids, gases, solids and surfaces. More specifically, the methodology of the present invention facilitate the formation of highly reactive species created by light and the photochemical interaction of said light with a proprietary non toxic multi-component compound for photo-catalytic rapid reduction, oxygenation, and effective chemical chain reactions which swiftly mineralizes, and dissolve, eliminate, an inactivate said noxious species, and thus turn them into more innocuous manageable forms. Further more, the methodology of the present invention is using lasers

### SUMMARY OF THE INVENTION

The present invention relates to a method for the advanced oxidation of dangerous chemical and biological sources suspected in particular regions, comprising the steps of;

(a) spraying over said regions a cloud of gas, vapors, microdroplets, droplets, or bubbles formed from at least one liquid solution containing at least one type of photocatalitic oxidizing substance;

(b) directing across said cloud at least one high intensity beam of light having wavelength of between 220 and 390 nanometer for triggering said cloud thereby causing a catalyzed activation that releases free radicals of said oxidizing substance in order to react with said chemical or biological sources.

The method of the present invention is useful for protection of public metropolitan areas, air volumes, surfaces, or combinations thereof using advanced oxidation processes triggered by pulsed UV laser, and photo-catalytic bubbles, droplets, jets, or combinations thereof, wherein said photo-catalytic components include at least one oxidizing agent, fluorescent, or phosphorous, or glowing, or reflecting, shifting, or vibrating or combination element such that once said photo reactive, or photo-catalytic compounds interact with light, it could be traceable for monitoring, and verification of bio-dosimetric dose, and chemical dissociation dosimetric values required for adequate inactivation and reduction, or minimization of threatening eventualities or contamination, cross contamination in any predetermined liquids, gases, surfaces, and combination.

The method of the present invention may further comprise scanning the cloud with the beam of light, which is especially essential for large scale treatments wherein the cloud extends along wide area (and volume).

The beam of light used in the method of the present invention is preferably emitted from at least one laser unit, wherein the laser unit may be selected according to design and engineering considerations, from; solid state lasers, electrical discharge lasers, plasma driven lasers, semiconductor lasers, organic lasers, electron beam pumped lasers, free electron lasers, fiber lasers doped, or SASE/EA/FEL lasers, fiber lasers, diode pumped lasers, crystal based lasers, doped glass based lasers, FELs lasers, polymer lasers, PW/CW type lasers, quantum dote lasers, laser arrays, flush lamp pumped lasers, water lasers, photonic bandgap lasers, seeded or amplified lasers, time compressed or expanded lasers, q-switch type lasers, interactive harmonic lasers, acoustic-optical lasers, ultrasonic lasers, X-ray pumped lasers, Y pumped lasers, E-beam lasers, catalytic lasers, photoelectrocatalytic lasers, air lasers, ground stationary lasers, mobile and sub-miniaturized lasers, thin film type lasers, vapor lasers, water lasers, or photonic bandgapped lasers.

According to various preferred embodiments of the method of the present invention, the laser beam is being pulsed.

The pulses of the beam may be in duty cycle of between 0.1 and 50 percent, in pulse repetition rate of between 1 Hertz and 100 Gigahertz.

According to various preferred embodiments of the method, the beam is pulsed in short pulse duration having acoustic attack transient properties.

According to various preferred embodiments of the method the cloud contains bubbles of size useful for opto-acoustic interaction between a beam of light scanning the cloud and the acoustic vibrations of the bubble mantles caused by the hit of the beam, which in turn, resulting in a multidirectional scattering of the beam, reflecting it to a plurality of surrounding bubbles or droplets.

According to various preferred embodiments of the method the solution contains phosphoric or fluorescense substance reactant to the light beam, to the light beam reflections from surrounding bubbles or droplets, or to particles released during the catalyzed activation, and wherein said method further comprising the step of tracing the light glowing from said phosphoric or fluorescense substance in order to identify whether or what portions of said region have been covered with an activated oxidizing substance.

According to the various embodiments of the method of the present invention, wherein the photo-catalytic bubbles or droplets contain at least one component of fluorescent, or phosphorous type, spectroscopic data acquisition can be made, and the accumulated data provides for dosimetric value for inactivation of noxious species of biological or chemical origin, and thus the spectral distribution over a predetermined space, over a predetermined period of time can be calibrated against specie specific calibration standards so as to identify the relevant thresholds needed for protecting, and treating application in predetermined surface area, volume or combination thereof. Thus, according to additional preferred embodiments, the method is further comprising measuring and calibrating the acoustic energy that light creates, and the light that acoustic energy creates, in order for establishing bio-dosimetric values or adapting the energy dose required for mineralizing or oxidizing predetermined toxic chemical species or biological noxious species,

According to various preferred embodiments of the method is further comprising the step of automatically deflecting the beam of light across the cloud according to light reflection data acquired from the cloud during the catalyzed activation of the oxidizing substance.

According to various preferred embodiments of the method (especially in building regions, wherein a system using the method of the present invention can be integrated with the conventional water system of the building, and/or with other existing security systems of the building) the solution is produced in real time or in immediate proximity to a disinfection procedure that is being implemented in an infection suspected site, and said solution is being prepared directly into flowing water supplied to sprinklers or bubble generators that create the cloud, wherein oxygen or any other essential dissolvent materials, are being dissolved into said flowing water during their flow to the sprinklers or bubble generators.

According to the method, the concentration of at least one of the solution ingredients may be controlled by a computer and is case sensitive to the seriousness of dangerous chemical and biological sources under treatment.

The present invention further relates to a system for advanced oxidation of dangerous chemical and biological sources suspected in particular regions, comprising;

(a) means for the formation and distribution of a cloud of gas, vapors, microdroplets, droplets, or bubbles formed from at least one liquid solution containing at least one type of photocatalitic oxidizing substance, said means having at least one outlet for releasing or spraying said cloud;

(b) at least one laser unit generating light beam having properties that are useful for triggering said cloud, thereby causing a catalyzed activation that releases free radicals of said oxidizing substance in order to react with said chemical or biological sources.

The system according to the present invention may further comprise, according to various preferred embodiments, means for deflecting said light beam for scanning the cloud.

Said means for deflecting said light beam for scanning the cloud, may further cooperate with camera or sensor means for acquiring and for processing data which relates to light glowing from different portions of the cloud during its triggering or during its catalyzed activation, and with means for steering the scanning automatically according to said data.

The present invention further relates to a bubble generator for forming a cloud of bubbles for the advanced oxidation of dangerous chemical and biological sources suspected in particular regions, comprising; at least one liquid container adapted to receiving a solution containing at least one type of photocatalitic oxidizing substance, means for gas compression, and at least one nozzle in liquid communication with the liquid reservoir, and in gas communication with the gas compression means.

The bubble generator may further comprise means for dissolving pure oxygen or any other essential dissolvent materials, into water flowing to the nozzle, for real time accomplishment of the solution.

The present invention further relates to a use of the system for the advanced oxidation of dangerous chemical and biological sources suspected in particular regions such as outdoor regions; domestic regions; sea, air or ground vehicles; or space ships, and to the use of the system for disinfection of medical and surgical equipment and spaces, and to the use of the system for disinfection of toilet rooms, wherein the system is integrated to the toilet room and having means for automatic activation, cooperating with sensor means for determining the absence of humans when activating a disinfection treatment sequence.

The present invention disclosed a novel methodology for Non destructively sealing, disinfecting, purifying, sterilizing wide varieties of medical instrumentation by inactivating noxious species on the surface of said instruments in wide variety of medical, commercial, industrial, domestic, and agricultural applications. Further more, the novel methodology of the present invention does not require any direct contact with the actual surface, and/or volume of liquids, and gases (containing or holding permanently, or temporarily to be disinfected).

More, specifically, the present invention combine the utilization of light which have the appropriate wavelength to penetrate the surface of the packaging materials which are used for enclosure, using the packaging itself for geometrical utilization guiding, and concentrating light therein (example: in the bottle FIG. 1, 1-30). Safely sealing, and sterilizing, disinfecting the liquids, and gases therein (example: in the bottle). and is calibrated for specie specific penetration spectrum or calibration standards against noxious species, such as found in water, and air, and/or liquids and gases, forming essential combination such as needed for sustaining life on earth. The present invention disclosed a novel methodology for disinfection through packaging selected from at least one PET packaging, or polyolefin, or polyamide, or polycarbonate, or polyesteramide, or polyester or any resine combination thereof having refractive index profile for partial, or total internal reflection therein for equalizing the concentration of bacteria, viruses, cysts, phatogens, or biological, or organic, non organic, or toxic or noxious species therein for example in packaging composed of selected materials such as at least one PET packaging, or polyolefin, or polyamide, or polycarbonate, or polyesteramide, or polyester or any resine combination thereof having refractive index profile appropriate for partial, or total internal reflection or homogenic scattering therein for the purpose of equalizing the concentration of bacteria, viruses, cysts, phatogens, or biological, or organic, non organic, or toxic or noxious species therein. The present invention discloses a novel methodology for disinfection through the packaging by using a UVA pulsed laser, or a continues laser, or hybrid interaction between CW, and PW types of light wherein said light is transferred, coupled to, or distributed through (packaging) materials selected from Polyolefine, polyamide, polycarbonate, polyesteramide, polyester resine combination thereof, or terephtalate (PET), Polybutylen terephtalate (PBT), polyalkyl terephtalate, polyethylene naphtalate (PEN), polyalkyl naphtalate (PETG) having adequate refractive index profile therein for guiding light throughout. Especially beneficial for external non invasive treatment methodology for reaching and geometrically utilizing small, and larger blood vessels (while still un effected in the body), and areas of the body wherein vital fluids needs to be treated in real time. Such beneficial embodiments and devices using the methodology of the present invention may be best described as optronic dialysis of liquids in real time without puncturing the external surface of the body, or volume to be disinfected, or treated. Thus optronic inactivation of specific species in blood may be facilitated using the methodology of the present invention. More specifically by using the methodology of the present invention end users could simply wear breaslates, and O/F wristlets, and specific devices using the method of the present invention delivering appropriate dose response curves for a predetermined specie specific calibration standards, thus allowing the determination of dose response curves for specific diseases, viruses, cysts, bacteria, pathogens, and noxious species. thus increasing beneficial sustaining, and delaying of the hydroxyl radial species lifetimes, increasing the quantum yield of efficiency according to the methodology of the present invention, especially beneficial for catalytic, scintillated, pH stabilized, oxygen charged toothpaste, or spray for the reduction of humic acids, and other factors effecting bad smells, tastes, substantially improving catalytic efficiencies of the methodology of the present invention.

The present invention relates also to a method for sterilizing and disinfecting the surface of medical instrumentation, comprising: Preferred mode of utilization for dentistry, general dentists, periodontists, prothodontists, pedodontists, pediatric dentists, endodontists, oral/maxillofacial surgeons, and orthodontists wherein the duty cycle of procedures thus performed in these respective field when using multicomponent catalytic U.PW based compound or coupling gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

More specifically, the methodology of the present invention also relate to a catalytic tooth paste capable of scintillating or repeatable triggering by using visible, UVA, UVB, UVC, optical triggering signal eliminating the need for repeatable brushing. More specifically the present invention facilitate the dissolving of pluck formation catalitically, scintillating, and deep disinfection, and dissociation therapeutic effective treatment.

In the context of the present invention Acquired immunodeficiency Syndrome means A life threatening disease caused by virus and characterized by breakdown of the body's immune defenses, specie specific calibration standards mean in the context of the present invention the efficient reduction of viruses which causing immunodeficiency syndromes without interfering with the body external surfaces.

In the context of the present invention Active immunity means that the immunity produced by the body in response to stimulation by a disease causing organism or vaccine, or treatment according to the present invention wherein blood is circulated in the body, or in loop to/from the body through optronic dialysis according to the methodology of the present invention and therapeutic responses are following as a result of the treatment according to the methodology of the present invention.

In the context of the present invention Agamaglobulinemia means An almost total lack of immunoglobulines, and/or antibodies.

In the context of the present invention Allergen means any substance that causes an Allergy, and/or allergic reaction by the misfunctioning of the MHC type 1, 2, 3, and/or any combination thereof.

In the context of the present invention Allergy means An inappropriate and harmful response of the immune system to normally harmless substances (i.e. see Allergic reaction as an example)

In the context of the present invention Anaphylactic Shoch a life threatening Allergic reaction characterized by swelling of the body tissues including the throat, difficulties in breathing, and a sudden fall in blood pressure.

In the context of the present invention Anergy means A state of unresponsiveness, induced when the T cell antigen receptor is stimulated, that effectively freezes T cell responses, pending a 2ND Signal from the Antigen presenting cell.

In the context of the present invention Antibody means a soluble protein molecule produced and secreted by B cell in the response to an antigen, which is capable of binding to the specific antigen.

In the context of the present invention Antibody-dependent Cell—mediated Citotoxicity (ADCC) means in the context of the present invention An immune response in which antibody, by coating target cells makes them vandurable to attack by immune cell (see coating and marking cells)

In the context of the present invention Antigen means any substance that, when in traduced into the body is recognized by the immune system.

In the context of the present invention Antigen presenting cells means B cells of the monocyt lineage (including macrophages as s dantritic cells), and various other body cells that present antigen in a form that T cells can recognize.

In the context of the present invention Anti nuclear antibody (ANA) means an autoantibody directed against a substance in the cell nucleus.

In the context of the present invention Antiserum means a Serum that contains antibodies.

In the context of the present invention Antitoxins mean Antibodies that in terlockk with and inactivate toxins produced by certain bacteria.

In the context of the present invention Appendix means a lymphoid organ in the intestine.

In the context of the present invention Attenuated means a weakened; no longer infectious (innocuous form)

In the context of the present invention Autoantibody an antibody that reacts against persons own tissue.

In the context of the present invention Auoto immune desease a desease that result when the immune system mistakenly attacked the bodies own tissues. For example the Rheumatoid arthritis and systemic lupus erythematosus aree auto immune diseases.

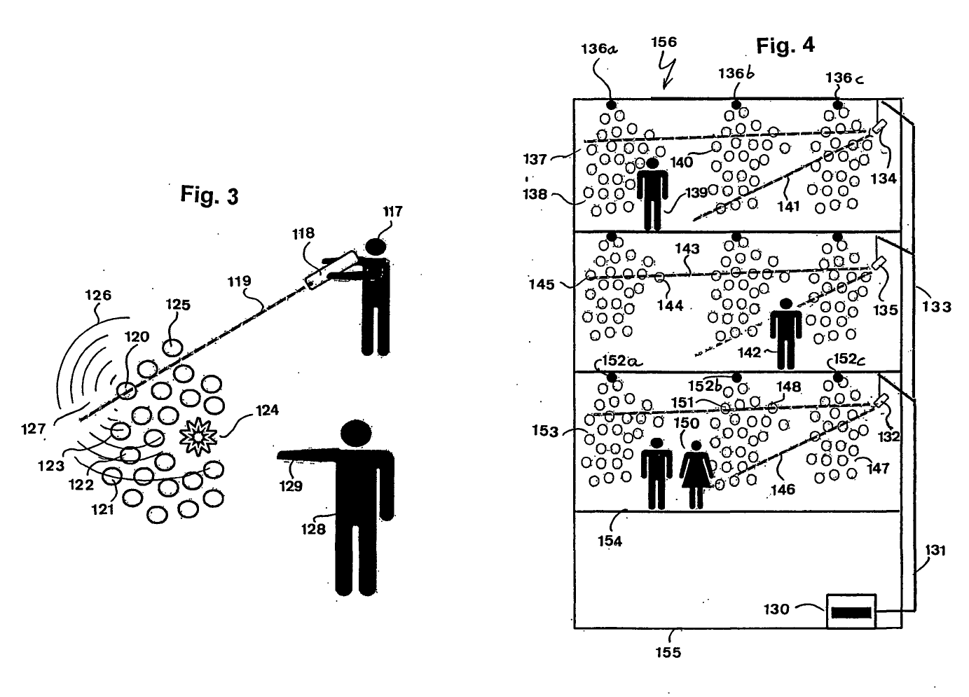

Especially beneficial for disinfection through the skin of internal blood flow, and bodily fluids without causing any damage, such as in an optical dialysis according to the present invention (non invasively ref: claim 1-100, FIG. 3) or for packaging materials by using UVA pulsed laser, operating in pulsed mode, beneficial for quality control applications (in mass production lines of mineral waters, flavored waters, beverages, juices, liquids, gasses, food based products, and medical preparation sites), and for optical, and electrooptical dialysis devices using the method of the present invention.

Including the production, and polishing of insulin based products. The novel methodology of the present invention is also beneficial for disinfecting corks, lids, and bottle necks of packaging, further more, the method of the present invention could be used to treat non invasively blood flow while in the body, by using pulsed high intensity UVA laser delivering a plurality of micropulses, leading to cumulative dose response delivered by using optical waveguides, and uniform diffusers.

In treating medical instrumentation and engineering tools with pulsed UVA, UVB, UVC laser light—where the geometrical curvature is often difficult to reach using conventional disinfection, or purification technologies. The method of the present invention is also disclosing a novel methodology wherein the cork, or lid themselves are made of polymer varieties having higher refractive index then the liquids, and/or gases therein (i.e. in the packaging), in order to maximize geometrical utilization.

More specifically, as an example; the methodology of the present invention for surface treatment is also beneficial for improving the hygiene of the mouth by harnessing the illumination or irradiation of a waveguiding dielectric brush [WDB] sterilizing its complex curvature inner surfaces, and volumes, having variable depth of penetration catalytically using new generation of paste, comprising:

Constructing or integrating a multi-component compound structurally modular, containing predetermined portion of yielding Oxygen Charge (SYOCH) in a U.P.W, PH stabilized, held temporarily or permanently in a 3D polymeric frame work of biodegradable biocompatible carbomer or BI-polymers expanded to contain photo-catalytic, and, or centilating conversion elementally each having predetermined electron charge transfer co.-efficiencies and absorption, refractive index profile, and acoustic properties, selected pre-production for quantum objective application specific efficiencies driven thus from supper conductive, to dielectric, or semi conducting, wherein the flexibility of water may provide generic structurally yielding Oxygen Charge accommodating into manageable forms the decomposed species inactivated radically (SYOCH1), within water, liquid or gas or air suspension, body fluids, or inside mouth using new generation of paste according to the methodology of the present invention.

The present invention relates also to a method for treatment of surfaces having complex curvature wherein additional substances may be added for electronic trapping so as to facilitate prolonging the lifetime of free radical species, OH, produced according to the methodology of the present invention, such substances may be selected from liquids, gasses, or solids, in order to trap electron-pair holes,

In the context of the present invention Bacterium means a microscopic noxious microorganism composed of a single cell. Mant but not all bacteria cause desease.

In the context of the present invention Bassophile means A white blood cell that contributes to inflammatory reactions. A long with mast cells, Basophiles are responsible for the symptoms of Allergy. (and/or Allergic reactions).

In the context of the present invention B cells means small white blood cells crucial to the immune defenses. Also known as B lymphocites, they are derived from bon Marrow and develop into plasma cells that are the source of antibodies.

In the context of the present invention Biological response modifiers means a substances, either natural or synthesized, that boost, direct, or restore normal immune defenses. BRMs include interferons, interleukins, thymus hormones and monoclonal antibodies.

In the context of the present invention Biotechnology means the use of living organisms or their products to make or modify a substance. Biotechnology includes recombinant DNA technique (such as genetic engineering), and hybridoma technology.

In the context of the present invention Bon-marrow means soft tissue located in the cavities of the bones. The bone marrow is the source of all blood cells.

In the context of the present invention Cellular immunity means immune protection provided by the direct action of the immune cells (as distinct from soluble molecules such as antibodies).

In the context of the present invention Chromosomes mean physical structures in the cell's nucleus that have the genes. Each Human cell has 23 pares of chromosomes.

In the context of the present invention Clone A group of genetically identical cells or organisms descended from a single common ancestor., (v) to reproduce multiple identical copies.

In the context of the present invention Complement means a complex series of blood proteins whose action complements the work of antibodies. Complement destroys bacteria produces inflammation, and regulates the immune reactions.

In the context of the present invention Complement cascade means a precise sequence of events usually triggered by an antigen/antibody complex in which each component of the complement system is activated in turns.

In the context of the present invention Constant region means that part of an Antibody structure that is characteristic for each antibody class.

In the context of the present invention Co stimulation means the delivery of a 2nd signal from an antigen-presenting cell to a T cell. The 2nd signal rescues the activated T cell from Anergy, allowing it to produce the lymphokinds necessary for the growth of additional T cells.

In the context of the present invention Cytikines means powerful chemical substances secreted by Cells. Cytokines include lymphokines produced by lymphocytes and monokines produced by monycites and macrophages.

In the context of the present invention Dandritic cells means] white blood cells found in the spleen and other lymphoid organs. Dandritic cells typically use thread like tentacles to enmesh antigen, which they present to T cells.

In the context of the present invention DNA (deoxyribonucleic acid) means a nucleic acid that is found in the cell nucleus and that is the carrier or co. represents genetic information. (I.e. see RNA, Ribonucleic Acid or any combination thereof)

In the context of the present invention Enzyme means a protein; produced by living cells that promotes the chemical processes of life without itself being altered.

In the context of the present invention Eosinophil means a white blood cell that contains granules filled with chemicals damaging to parasites, and enzymes that damp down inflammatory reactions.

In the context of the present invention Epitop means a unique shape or marker carried on antigen's surface, which triggers a corresponding antibody response.

In the context of the present invention Fungus means a member of a class of relatively primitive vegetable organism. Fungi include mushrooms, yeast, rusts, molds and smuts.

In the context of the present invention Gene means a unit of genetic material (DNA) that carries the directions a cell uses to perform a specific function, such as making a given protein.

In the context of the present invention Graft-versus-host disease (GVHD) means a life threatening reaction in which transplanted immunocompetent cells attack the tissues of the recipient (such as in transplant medical procedures).

In the context of the present invention Granulocytes means A white blood cell file with granules containing potent chemicals that allow the cell to digest micro organisms (noxious types and non noxious types) or to produce inflammatory reactions. Neutrophiles eosinophiles and basophiles are examples of granulocytes.

In the context of the present invention Helper T cells means A subset of T cells that tipically carry the T4 marker and are essential for turning on the antibody production, activating cytotoxic T cells, and initiating many other immune responses.

In the context of the present invention Hematopoiesis means the formation and development of blood cells, usually takes place in the bone marrow.

In the context of the present invention Histo Compatibility Testing means A method of matching the self-antigens (HLA) on the tissues of a transplant doner with those of the recipient. The closer the natch, the better the chance that the transplant procedure will be successful “and will take”

In the context of the present invention HIV means (Human Immunodificiancy virus) it is the virus that causes AIDS.

In the context of the present invention Human lococyte antigens (HLA) means a protein in markers of self use in histocompatibility testing. Soma HLA types also correlate with certain autoimmune diseases.

In the context of the present invention Humoral immunity means an immune protection provided by the soluble factors such as antobodies, which circulate in the body fluids or Humors, primarily Serum and lymph.

In the context of the present invention Hybridoma means A Hybrid cell created by fusing B lymphocytes with as long lived neoplastic plasma cell or T lymphocyte with a lymphoma cell. B cell Hybridoma secretes a signal specific antibody.

In the context of the present invention Hypogammmaglobuilinemia means is an abnormally low level of immunoglobuline.

In the context of the present invention Ideotypes means a unique and characteristic part of an antibody variable region, which can dam cells serve as antigens.

In the context of the present invention Immune Complex (IC) means a Cluster of interleukines, antigens and antibodies.

In the context of the present invention Immune response means the reactions of the immune system to foreign substances.

In the context of the present invention Immunoassay means A test using antibodies to identify and quantify substances. Often the antibody is link to a marker such as a fluorescent molecule, a radio active molecule or an enzyme type, or combination of the above

In the context of the present invention Immunocompetent means the capability of developing an immune response.

In the context of the present invention Immunoglobulines means a family of large protein molecules also known as antibodies.

In the context of the present invention Immunosupression means A reduction of the immunresponses for instance by giving drugs to prevent transplant rejection/s.

In the context of the present invention Immunotoxines A monoclonal antibody linked to a natural toxins, a toxic drugs or radioactive substance or combination.

In the context of the present invention Inflammatory response means A redness, warmth, swellin g, pain, and/or loss of function produced in response to infection as the result of increased flood flow and an influx of immune cells and secretions.

In the context of the present invention 18nterleukines means A major group of lymphokines and monokines.

In the context of the present invention Kupffer cells means specialized macrophages in the liver.

In the context of the present invention LAK cells means lymphocytes transformed in the laboratory into lymphokines activated Killer cells, which attack tumor cells.

In the context of the present invention Langerhans cells means A drandritic cells in the skin that pick up antigen and transport it to lymph nodes.

In the context of the present invention Leukocytes and all white blood cells.

In the context of the present invention Lymph means a transparent, slightly yellow fluid that carries lymphocytes, bathes the body tissues, and drains into the lymphatic vessels.

In the context of the present invention In the context of the present invention Lymphatic vessels mean a body-wide network of channels, similar to the blood vessels, which transport lymph to the immune organs and into the bloodstream.

In the context of the present invention Lymph nodes mean small bean-shaped organs of the immune system, distributed widely throughout the body and linked by lymphatic vessels. Lymph nodes are garrisons of B, T, and other immune cells.

In the context of the present invention Lymphocyte means small white blood cells produced in the lymphoid organs and paramount in the immune defenses.

In the context of the present invention Lymphoid organs mean the organs of the immune system, where lymphocytes develop and congregate. They include the bone marrow, thymus, lymph nodes, spleen, and various other clusters of lymphoid tissue. The blood vessel and lymphatic vessels can also be considered lymphoid organs.

In the context of the present invention Lymphokines means powerful chemical substances secreted by lymphocytes. These soluble molecules help direct and regulate the immune responses.

In the context of the present invention Macrophages mean a large and versatile immune cell that acts as a microbe-devouring phagocyte, an antigen-presenting cell, and an important source of immune secretions.

In the context of the present invention Major histocompatibility complex (MHS) means a group of genes that controls several aspects of the immune response. MHC genes code for self-markers on all body cells.

In the context of the present invention Mast cells means a granule-containing cell found in tissue. The contents of mast cells, along with those of basophils, are responsible for the symptoms of allergy.

In the context of the present invention Microbes means minute living organisms, including bacteria, viruses, fungi and protozoa.

In the context of the present invention Microorganisms means microscopic plants or animals.

In the context of the present invention Molecule means the smallest amount of a specific chemical substance that can exist alone. (The break a molecule down into its constituent atoms is to change its character. A molecule of water, for instance, reverts to oxygen and hydrogen).

In the context of the present invention Monoclonal antibodies mean antibodies produced by a single cell or its identical progeny, specific for a given antigen. As a tool for binding to specific protein molecules, monoclonal antibodies are invaluable in research, medicine and industry.

Monocyte means a large phagocytic white blood cell which, when it enters tissue, develops into a macrophage.

In the context of the present invention Monokines means powerful chemical substances secreted by monocytes and macrophages. These soluble molecules help direct and regulate the immune responses.

In the context of the present invention Natural killer (NK) cells mean large granule-filled lymphocytes that take on tumor cells and infected body cells. They are known as natural killers because they attack without first having to recognize specific antigens.

In the context of the present invention Neutrophil means a white blood cell that is an abundant and important phagocyte.

In the context of the present invention Nucleic acids means large, naturally occurring molecules composed of chemical building blocks known as nucleotides. There are two kinds of nucleic acids, DNA and RNA.

In the context of the present invention OKT3 means a monoclonal antibody that targets mature T cells.

In the context of the present invention Opportunistic infection means an infection in an immunosuppressed person caused by an organism that does not usually trouble people with healthy immune systems.

In the context of the present invention Opsonize means to coat an organism with antibodies or complement protein so as to make it palatable to phagocytes.

In the context of the present invention Organism means an individual living thing.

In the context of the present invention Parasite means a plant or animal that lives, grows, and feeds on or within another living organism.

In the context of the present invention Passive immunity means immunity resulting from the transfer of antibodies or antiserum produced by another individual.

In the context of the present invention Peyer's patches means a collection of lymphoides tissues in the intestinal tract.

In the context of the present invention Phagosites means a large white blood cells that contribute to the immune defenses by ingesting microbes or other cells and/or forign particles.

In the context of the present invention Plasma cells means a large antibody producing cell that develops from B cell.

In the context of the present invention Platelets mean A granule containing cellular fragments critical for blood clotting and sealing of wounds. Platelets also contribute to the immune response.

In the context of the present invention Plymorphes means short for Poly Morpho Neuclear locosytes or granulocytes.

In the context of the present invention Proteins means an organic compound made up of aminoacids. Proteins are one of the major constituents of plants and animals and human cells.

In the context of the present invention Protozoa means a group of one-celled animals few of, which can cause Human desease (including maleria and sleepoing sickness types)

In the context of the present invention Rheumatoid factor means an auto antibody found in the serum of most persons with rheumatoid arthritis.

In the context of the present invention RNA (Ribonucleic Acid) a nucleic acid that is found in the cytoplasm and also in the nucleus of some cells. One function of RNA is to direct the synthesis of proteins.

In the context of the present invention Scavenger cells means any of the diverse groups of cells that have the capability to engulf and destroy foreign materials, dead tissues, or other cells.

In the context of the present invention SCID mouse a laboratory animal that, lacking enzyme necessary to fashion an immune system of its own, can be turned into a model of the human immune system when injected with human cells or tissues.

In the context of the present invention Serum means a clear liquid that separates from the blood when it is allowed to clot these fluids retains any antibodies that were present in the whole blood.

In the context of the present invention Severe combined immunodefficency desease (SCID) means A life threatening condition in which infants are born lacking all major immune defenses.

In the context of the present invention Splin means a lymphoid organ in the abdominal cavity that is important center for immune system activities.

In the context of the present invention Stem cells mean Cells from which all blood cells are derived. The bone marrow is rich in stem cells.

In the context of the present invention Suunit vaccine means a vaccine that uses marely one component of an infectious agent rather then the whole to stimulate an immune response.

In the context of the present invention Supeantigens means a class of antigens, including certain bacterial toxins that unleash a massive and damaging immune response.

In the context of the present invention Suppresser T cells means a subset of T cells that turn off antibod y production and other immune response.

In the context of the present invention T cells means small white blood cells that orchestrate and/or directly participate in the immune defenses. Also known as T lymphocytes, they are processed in the thymus and secret lymphokines.

In the context of the present invention Thymus means a primary lymphoides organ, high in the chest where T lymphocyte proliferate and mature.

In the context of the present invention TIL means a tumor Infiltrating Lymphocytes. These immune cells are extracted from a tumor tissue, treated in laboratory and re-injected into the cancer patient.

In the context of the present invention Tissue typing means (see MHC, Major Histocompatibility Testing HCT).

In the context of the present invention Tolerance a state of nonresponsivnes to a particular antigen or group of antigens.

In the context of the present invention Tonsils and adenoids means are a prominent oval massive of lymphoid tissues on either side of the throat.

In the context of the present invention Toxins means agents produced by plants and bacteria, normally very damaging to mammalian cells, that can be delivered directly to target cells by lionkiun gthenm to monoclonal antibodies or lymphokines.

In the context of the present invention Vaccine means a substance that contains antigenic components from an infectious organism by stimulating an immune response (but not disease) it protect against subsequent infection by that organism.

In the context of the present invention Variable region means that part of an antibody structure that differs from one antibody to another.

In the context of the present invention Virus means a sub-microscopic microbe that causes infectious disease. A virus can reproduce only in living cells.

In the context of the present invention Libido-pranic a state of increased vitality (i.e. the word prana, pranic means vitality, or states of ample vitality), or such states in which the body is tuned, and the immune system is at rest, empowered by biological traces of specific origin, and nature. Libido is the sensual drive induced as a result of the body being tuned, and/or referenced, and systems in the body aren't over loaded, and thus a state of improved feelings is induced, i.e. a libido-pranic state.

In the context of the present invention Resonativistic means a state of resonance created when energy of high peak powers is being applied, projected into, coupled to, or are being generated into biomass, such resonance is subject to the expansion In the context of the present invention ration, or density of the holding media, or medium, the term represent a state in which light and sound, ultrasound and mechanical, and physiological process are occurring and causing resonance which when said resonance utilizes the individual resonance of elements, organs, or cells of the noxious species, and could identify, or recognize, separate, sort and inactivate, dissociate, or vibrate said biomass,

In the context of the present invention The following terms included to improve the understanding of photochemistry the specific context have been selected for benefits of clarity and familiarization.

Physical Constants of interest in Ultraviolet and Photochemistry means

| Constant | Symbol | Value | Units |
|---|---|---|---|
| Speed of light | c | 2.99792458 × 10⁸ | M s⁽⁻¹⁾ |
| Charge on | e | 1.60217733 × 10⁽⁻¹⁹⁾ | C |
| electron | | | |
| Planck constant | h | 6.6260755 × 10⁽⁻³⁴⁾ | J s |
| Boltzmann | k | k 1.380658 × 10⁽⁻²³⁾ | J K⁽⁻¹⁾ |
| constant | | | |
| Avogadro number | NA | 6.0221367 × 10²³ | mol⁽⁻¹⁾ |
| Gas constant | R | 8.31451 | J mol⁽⁻¹⁾ K⁽⁻¹⁾ |

Further Characteristics of light and context explanatory notes are herewith included:

In the context of the present invention Planck Law of Radiation means in the context of the present invention:

Light which has both particle and wave properties. It is transmitted in discrete packets of energy (photons) and yet has a frequency and wavelength. The connection between these two properties is embodied in the Planck flaw of Radiation

In the context of the present invention Photochemical wave changes means in the context of the present invention:

In the context of the present invention The usual wavelength range in Photochemistry is 100-1000 nm. Light photons with wavelengths longer than 1000 nm have a photon energy too Small to cause chemical change when absorbed, and photons with wavelengths shorter than 100 nm have so much energy that ionization and molecular disruptions characteristic of radiation chemistry prevail. The total In the context of the present invention photochemical wavelength range is divided up into bands with specific names as given below.

In the context of the present invention Spectral ranges or interest in Photochemistry means in the context of the present invention:

| Range Name | Wavelength |
|---|---|
| Near Infrared | 700-10000 |
| Visible | 400-700 |
| Ultraviolet | |
| UVA | 315-400 |
| UVB | 280-315 |
| UVC | 100-280 |

Little photochemistry occurs in the Near Infrared. Except for some photosynthetic bacteria. Which are capable of storing solar energy at wavelengths out to 980 nm. The Visible range is completely active for photosynthesis in green plants and algae. Also many dyes can undergo photochemical transformations themselves or sensitize reactions in other molecules. Most studies in photochemistry involve the Ultraviolet range. The division into three sub-ranges [UVA, UVB, UVC] is connected with the human skin's sensitivity to ultraviolet light. The UVA range causes changes in the skin that lead to sun tanning. The UVB range can cause sun burning and is known to eventually induce skin cancer. The UVC range is extremely dangerous since it is absorbed by proteins, RNA and DNA and can lead to cell mutations and/or cell death. The UVC range is sometimes called the germicidal range, since it is very effective in inactivating bacteria and viruses. The Vacuum Ultraviolet range is absorbed by almost all substances (including water and air). Thus it can only be transmitted in a vacuum. The absorption of a VUV photon causes one or more bond Breaks. However, even though photons with wavelengths less than 561.6 nm are capable of splitting the H202 molecule, no photolysis, or proteolysis occurs in this wavelength region because H202 does not begin to absorb ultraviolet light until below 300 nm. This illustrates the first Law of Photochemistry; namely that no photochemical reaction can occur unless a photon/s of light is absorbed.

In the context of the present invention Coherent and incoherent light means Light sources used in photochemistry can either be coherent (all emitted photons are in phase with each other as they propagate) or incoherent (all emitted photons have random phases). All lasers emit coherent radiation and usually at one wavelength. The dispersion is very small so that a laser beam remains at or near its original diameter as it propagates; the light emitted by all other light sources is almost always incoherent. Most of these sources are either “hot element” sources (e.g., the incandescent light bulb) or “plasma” sources (e.g., a fluorescent light tube).

In the context of the present invention Point sources, means Light sources have finite dimensions (e.g., often a cylindrical shape). Emission from such a source is difficult to treat mathematically. It is convenient to mode] these sources as a collection of point sources, in which all light is emitted from the point equally in all directions. The optics treatment for point sources is especially simple.

In the context of the present invention The Terms and concepts associated with the emission of tight, are herewith included for clarity of explanation and to simplify the understanding of the method of the present invention, especially wherein photochemistry is involved, or photochemical polishing is active in the processing according to the present invention:

The light emitted from a source can be viewed in many different ways. In this Section, the various terms that may be used to describe this emission are defined and explained.

In the context of the present invention Radiant energy means:

Radiant energy (Q) is a total amount of radiant emission (J) from a source over a given period of time,

In the context of the present invention Radiant power means:

The radiant power (P) of a source is the rate of radiant energy or total radiant power (W) emitted in all directions by a light source. For example, the radiant power of the Sun is 3-842×1026 w, in theory, P, should include all wavelengths emitted by the source; however, ˜ is usually restricted to the wavelength range of interest to photochemistry. For example, if a light source were being used for ultraviolet photochemistry, P would be specified for emission in the 200-400 nm ultraviolet ranges.

In the context of the present invention Radiant power efficiency means:

The radiant power efficiency (q) is defined as

Q=P/e

Where e is the input electrical power (W) supply.

Radiant emittance or excitance means:

The r Radiant emittance or excitance of a source is the radiant power emitted from an infinitesimal area on the surface of the source.

In the context of the present invention Radiant Intensity means:

The radiant intensity (I) (W sr\hat{ }(−1)) is the total radiant power P emitted by a source in a given direction about an infinitesimal solid angle.

Radiance means:

Radiance (L) is defined as the radiant power d²P, emitted from an infinitesimal area dA of the source surface in a given direction about the solid angle di, divided both by the solid angle di) and the orthogonal projected area.

The emittance M from an infinitesimal surface element dA is obtained by integrating L in spherical polar coordinates over the hemisphere of all outward-bound directions above dA.

An isotropic light source is defined as one in which the radiance L is uniform over all

Outward directions. Terms and concepts associated with the receipt of light When light is emitted from a source, it radiates outward at the speed of light, when it impinges on an object, it may be reflected, transmitted or absorbed. There are several terms that relate to the receipt of light.

Fluence Rate means:

Fluence Rate (E) (W m\hat{ }(−2) is the radiant power of all wavelengths passing from all

Directions through an infinitesimally small sphere of cross-sectional area d, divided by

CM

Irradiance means:

Irradiance (symbol E; units W m\hat{ }(−2) is defined as the total radiant power of wavelengths incident on an infinitesimal element of surface of area as containing the point under consideration divided by as. The following are some important points regarding characteristics and differences between “irradiance” and “fluence rate”:

Examples: For a parallel and perpendicularly incident beam, not scattered or reflected, irradiance and fluence rate becomes identical. For any UV source within a three-dimensional volume, the integration of UV irradiance over the interior surface of the volume yields the UV power of the lamp., This is not true for UV fluence rate.

The appropriate term for UV disinfection is “UV fluence rate” because a

Microorganism can receive UV power from any direction, especially when there is more than one UV lamp in the vicinity. In general usage, the irradiance or fluence rate may be expressed as MW cm\hat{ }(−2). The irradiance is often incorrectly termed “light intensity” see the proper definition of “radiant intensity” above.

Light dose or fluence means:

The light dose or fluence (symbol H, units J m˜2) is the total radiant energy of all

Wavelengths passing from all directions through an infinitesimally small sphere of cross-sectional area dA, divided by dA It is given by the average fluence rate times the exposure tune in seconds. The term UV dose is often used in UV disinfection literature. It represents the UV exposure of a given organism in the germicidal range.

Spectral units means:

All of the terms for tight emission or incidence refer to all relevant wavelengths. One can define spectral derivatives for each of these terms. For example, the light power emission of a LIV lamp is often expressed as the spectral power (W nm\hat{ }(−1), defined as the power output in a narrow wavelength band divided by the width of the band. The solar spectrum received at the Earth's surface is described in terms of the solar spectrum irradiance. Also the spectral distribution of a lamp emission is often given an a plot of spectral power versus wavelength.

Photon based units means:

Photochemistry involves the interaction of photons of light with molecules and means: the definitions units that are based on photons.

Photon irradiance, photon fluence rate and photon flow means:

Each of the spectral terms can be convened to a corresponding equivalent photon flow and fluence rate ' by dividing the term by the average photon energy in the narrow wavelength band.

Quantum yield means:

The quantum yield (unites) Q is a measure of the photon efficiency of a photochemical reaction, e Is defined as the number of moles of product formed or reactant removed (P) per Einstein of photons absorbed

Line sources means:

When atoms are raised to an excited state, they emit only in very narrow lines with

Virtually no emission between the lines. The low-pressure mercury lamp is a very Common lamp of this type. Table 3 gives the wavelengths and relative emittance for the emission lines of a low-pressure mercury vapor lamp.

Certain radiation units and associated light sources, (lasers) and lamps emit at longer wavelengths, This is the basis of the very popular fluorescent lamp.

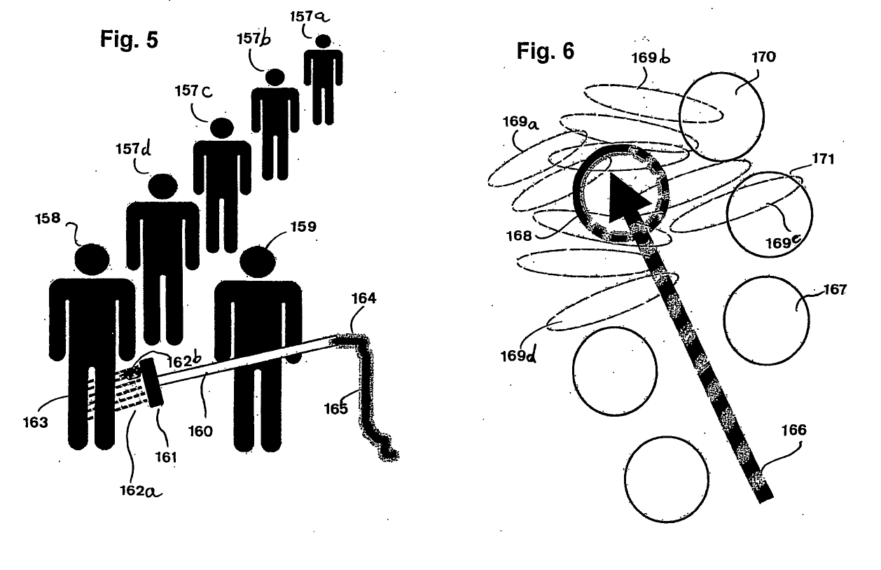

For example, The emission lines of a mercury lamp are only sharp when the pressure of the gas is low (<10 tore). If the pressure is increased, the lamp can carry much more power, but the emission lines broaden. For the same length of lamp (about 120 cm), a medium pressure lamp (pressure about 1000 tore) can carry up to 30,000 W. These lamps are very common in commercial Systems utilizing ultraviolet light. FIG. 5 shows a comparison of the emission of low pressure and medium pressure lamps in the ultraviolet region.

Excimer lamps means:

Excimer lamps arc unique in that they emit in a narrow band of wavelengths. An excimer is an atomic dimmer that is stable only in the excited state and dissociates on decaying to the ground state. Table 4 gives the wavelengths of sonic of the common excimer lamps.

Examples: Emission wavelengths (or some common excimer lamps

| Excimer | Wavelength (nm) | Excimer | Wavelength (nm) |
|---|---|---|---|
| Xe2 | 172 | XeCI | 308 |
| KrCI | 222 | I2 | 342 |
| CI2 | 259 | | |

Flash lamps

Flash lamps are similar to continuous wave (CW), but could also operate in (PW), pulsed mode operation, and are lamps that consist of a cylindrical quartz tube with electrodes at each end and filled with a ga.½ (e.g. xenon). A power supply “fires' the lamps by discharging a large amount of electrical energy in a very short period of time (several us) by applying a very high voltage (10-30 kV}. The resulting plasma reaches temperatures of 10,000-13,000 K and the emission is essentially that of a blackbody (see FIG. 4). In commercial flash lamp Systems the lamp, a typical “flashed” about 30 times per s, but given a special electronic pulsing circuitry is added, repetition rates could reach Khz regime.

FEL means Free Electron Laser and its derivatives, wherein space charged technologies (such as the Electrostatically Accelerated Free Electron Laser) include an electronic pulsing circuits, charging, or an accelerator (Such as R.F. Linac) is involved in the production of photons (around 100, 000,000 photons per electrons, in contradistinction of a conventional crystal based laser having around 1 photon per electron, it is a laser which have less maintenance associated with its operation and its wall plug efficiency is reaching around the 40-51% respectively, and respectfully of the exact pumping geometry used.

The term EAFEL means Electrostaticely Accelerated Free Electron Laser and is an extremely efficient laser pumping geometry wherein recycling of the Accelerated electrons is being performed by utilizing deceleration techniques and its wall plug efficiency is estimated to reach in access of the 55% (amount of light being produced or converted from the electricity being consumed for its operation.

Biologically enhanced or photo-chemically polished drinking water or breathing air means any liquid or gas which have been passed through or processed by the method of the present invention such as water and/or air), more specifically such processes involved in the polishing and enhancing may include; Optical inactivation, disinfection, inactivation of DNA and/or RNA replication sequences, Photocatalysis, electro catalysis, a hybrid of photo and electro-catalysis, optical dissociation, physiological dissociation, biomass expansion, filtration (pre/post), physical separation and sorting, reactivation, activation, sonication, acoustics, electroacoustics, electro-optical treatment (by photons of light), transgressing, or transversing said liquids and gasses through a photonic band gaped wave guides having an aerobic, non toxic passage for light and liquids or gasses or combination together, synchronously and or separately.

Peak power means, the energy generated when squeezing (i.e. such as when pulsing) electromagnetic energy in short duration of time, for example: a pulse of a given average energy and power—lasting, or having a pulse width of around 1 second (1s) will generate several watts in peak power, a pulse lasting or having width of microseconds (ms) will generate peak powers reaching the kilo-watts scale, while a pulse lasting nano (ns) seconds will generate peak powers reaching into the hundreds of million of watts which is especially beneficial for purposes such as optical dissociation, optical inactivation, optical polishing, and optical secretion and spectroscopy for control and diagnostics, so in short the shorter the pulse duration the higher its respective peak power.

Multi-photon—absorption—processes means a processes which when harnessed could be very beneficial for the photochemistry involved in the processing according to the present invention, for example when 10 mj of energy (250,000 photons) are projected into a liquid or gas, the time it takes this projection is very important, if these photons will be furnished over 1 second time domain, then it leaves sufficient time for the electrons in said liquid or gasses, to relax back to the relaxed state, but if we apply these photons in a time domain of 5 nano seconds, then we do not leave time for the electrons to relaxed and the processes is called Multiphoton absorption processes, this processes are non linear in nature and yield much higher quantum yield, or efficiencies, or speed of the reactivation, or a more efficient methodology for optical treatment, processing and polishing

A hybrid of light sources means plurality of light sources and wherein their total spectral emitence, or total spectral distribution, or their total irradiance will cause multiphotonic absorption processes by means of super imposing their time domain (example: 1 light source is slow=1s pulse duration, and additional light sources are very fast=5 ns laser for example), their total irradiance is great and beneficial for the processes of the method of the present invention to occur efficiently, further more, such hybridization could includes, lamps and lasers, lasers and flash lamps, or any combination of CW, or PW type of light source working together, in synchronisity, and or sequentially or link or resolved by time domain manipulation for maximizing photonic interaction in matter, especially beneficial for triggering th catalytic scintillating elemental compound according to the present onvention.

Photo catalysis means: The use of energy of a photon of light to catalyze chemical reactions. More specifically, such reaction may include the decomposition of water into hydrogen, and oxygen, and the complete oxidation of organic contaminants in aqueous environments. More specifically, the first step in Photocatalysis is for the catalyst material to absorb photon of light in order to excite an electron from the velance band (VB), to the conduction band (CB), thus creating electron-hole-pairs. Each Species must then migrate to the surface before recombination occurs. If this condition is met, the electron can be transferred to a surface adsorbed molecule, reducing it. The overall process is illustrated, it is important to note that for the processes to occur efficiently (preventing pre-matured recombination of the said electron-hole pairs), the rates of reduction, and the oxidation must be comperable. The position of the band edges is critical for each step of the process, a photocatalyst material which is stable in water is tio2,(or known as Titanium Oxide).

Electrocatalysis, similar to that explained in photo-catalysis, but instead of photons, an electrical charge is used, through the use of semiconductor material which has been specially selected (its band gap) for the charge applied, for the context of the present invention an electrocatalysis, stable in water is I.T.O, or known in its chemical name and signature Indium Tinoxide). Further more, it is especially beneficial to combine, and operate both electrocatalysis, and photo-catalysis simultaneously, or serially, or sequentially or in unison, or each separate catalytic is triggered separately in order to maximize the collective efficiencies, thus harnessing and improving the performance of current and future catalytic technologcal evolution according to the methodology of the present invention.

### DETAILED DESCRIPTION OF THE INVENTION

The present invention disclosed a novel methodology for non-invasive disinfection, purification, and inactivation or equlization of (DNA, & RNA) replication sequences of noxious species in myriad of biomedical, and biotechnological applications involving end users, producers, and researchers in associated fields comprising the following steps:

1) Filling, distributing into, holding, or storing—in a predetermined chamber or conduit, a predetermined volume of liquids, or gasses to be non-invasively non residually treated.

2) Closing said conduit, or chamber with a transparent polymer or glass type lid, having a predetermined action spectrum, able to deliver, and transmit throughout any wavelength of light from about 260 nm, to about the 360 nm.

Implementation of the novel methodology of the present invention for surface treatment and for improving the hygiene of the mouth, may include (a) harnessing the illumination or irradiation of a waveguiding dielectric brush [WDB] having delivery capacity from about one quarter of a million of photons per Cm²/Second, to about 999 trillion potons per Cm²/1 picosecond, or Femtosecond, or Atosecond, thus swiftly sterilizing its complex curvature inner surfaces, and volumes, having variable depth of penetration catalytically using new generation tooth paste comprising; Catalytic centilayted compound [CCC] according to the methodology of the present invention, and wherein

Constructing or integrating a multi-component compound structurally modular, containing predetermined portion of yielding Oxygen Charge (SYOCH) in a U.P.W, PH stabilized, held temporarily or permanently in a 3D polymeric frame work of biodegradable biocompatible carbomer or BI-polymers expanded to contain photo-catalytic, and, or centilating conversion elementally each having predetermined electron charge transfer co.-efficiencies and absorption, refractive index profile, and acoustic properties, selected pre-production for quantum objective application specific efficiencies driven thus from supper conductive, to dielectric, or semi conducting, wherein the flexibility of water may provide generic structurally yielding Oxygen Charge accommodating into manageable forms the decomposed species inactivated radically (SYOCH1), within water, liquid or gas or air suspension, body fluids,or inside mouth.

The present invention discloses a novel methodology for treating surfaces with changing curvature parameters. More specifically, by photocatalytically innitiating protection for medical instrumentation by light, the methodology according to the present invention facilitate the formation of a catalytic light barrier technology creating a “fire wall” wherein no known noxious specie may penetrate. The present invention a also disclosed a novel methodology for treating surfaces with changing curvature parameters. More specifically, as an example; by photocatalytically initiating protection for inner space of the mouth by light, the methodology according to the present invention facilitate the formation of a catalytic light barrier technology creating a “fire wall” wherein no known noxious specie may penetrate, replicate, and thus have no ability to infect (inactivation of their DNA, and RNA replication sequences). Especially Beneficial for the removal of plaque formation in periodontal treatment, and in keeping maintaining, and reapeatingly triggering photocatalytically, centilating processes, thus oxidizing noxious species which may inhibit the surfaces curvature, and the deeper layers, and avolume surrounding the mouth area.

More specifically, wherein cuts and sores to body external surface, may cause damage to external body parts/tissues, and thus open the potential for infecious events entering the body, such events introduce potential threat of contamination, and cross contamination through use, and thus the methodology according to the present invention offer the realization and harnessing of advance catalytic oxidation technology facilitating uncontestable technological advantages.

More specifically by prepering in advance all relevant parameters for efficient oxidation processes to occur (i.e. such as Oxygen, PH levels, stebilization, and the photocatalysts) in a tight cohesive, and homofenic multi-component system thus the present invention simplify, and gurenties photocatalysis processes in the presence of triggering energy density thresholds. The present invention discloses competitive advantages when geometrically utilizating thouroughlly space, and time, and light causing sterilization by pulsed ultraviolet (time domain driven) laser light for disinfection of wide variety of medical instrumentation and engineering tools.

Several technologies exist for the provision of surface treatment applications, These currently used technologies (x) introduce strict safety, reliability, and credibility and efficiency limitations due to their chemical, residual, often toxic, expensive, slow, labor and material driven procedures. Further more current methodologies for surface treatment of instrumentation is cumbersome, and could not easily be adapted to cover applications requiring actual treatment of physiological damage to tissues, and/or for cuts, sores, wounds, to living human beings. Further more, in the field, or as in during critical medical procedures under time constraints, wherein often thereisn't sufficient time available to wait for certain chemical action to proceed (such as when using biocydal, or chemical disinfectants), or for instrumentation to be returned from central, often far autoclaving, and disinfection equipment centre (i.e. such as in hospitals, and medical centres, or clinics), Faile to provide adequat safety measures for vital tools, and instrumentation, often results in making their associated working cycles longer, less efficient, requiring substantial replacement hardware components, and leads to unessary manual procedures, and subsequent expenses in human resources, and high energy consumption (high capital, and operation costs) as well as potential falure of vital medical procedures. The methodology according to the present invention is not so limited, that's why the present invention could be utilized for wide variety of application including but not limited to (a) Ultra sound procedures (b) medical surgical procedures, (c) Dental treatment procedures, (d) Cosmetic procedures, (e) Gynicological procedures, (f) 1^{st} aid treatment applications, (g) Bulk sterilization of tools and medical instruments, (h) Medical preperation, (I) Transplant procedures, (j) Diagnostic procedures, (k) By-pass operations, (I) Skin, and darmatological treatment procedures, (m) Chemical production sites for drugs, and medicine (n) Rehabilitation centres, (o) Hospitals, (p) Clinics, (q) Operation procedures in cancer treatment, (r) Mediacal procedure for birth, and pregnency diagnostics, and treatment, (s) Treatment of burns, (t) Treatment of cuts, bruises, wounds, (w) Treatment of areas exposed to radiation, (x) Treatment of packaging of medical preperation, and drugs production or analysis, (y) Treatment of surgical instrumentation, (z) Treatment of diabitis wounds.

The present invention is also beneficial for cleaning applications such as (100) Cleaning vehicles, (101) Cleaning airplanes, (102) cleaning ships, and (103) busses, (104) lories, and (105) semi-trailers, (106) tankers on land, sea, and (107) air. Further more, several applications illustrated herewith as having best mode for utilization of the methodology of the present invention.

More specifically, Such preffered applications clearly illustrate an important inovative solution wherein the size of the actual triggering light (or energy, and/or energy density under no circumstances dictate the size of the catalytic ‘fire”, or free radical species thus generated by the processes according to the presesnt invention. The present invention disclose a novel methodology for hybrid disinfection of surfaces, to disinfection of volumes, to static storage sites, to large installations treating high flow rates or complexed surface curvatures (i.e. 2D/3D) such as apearing to shape modern medical instrumentations, and wide varieties of engineering tools.

a/1 Air based catalytic compound oxygen charged and triggered, b/1 Catalytic globulin mixture wherein its best mode of being trigered is by at least one pulse of light being generated by a high peak power, high repetition rate laser, c/1 A catalytic compound lighter, or heavier then air being triggered while in transition or drifting, and wherein its catalytic actions produces sufficient free radicals to efficently dissolve toxic substances, traces, or any liquid or gas or combination containing noxious or poisonous nature, d/1 Steam type catalytic multi component compound having being produce on the ground or from a flying vhiecle or plain, or propeled to/from different locations according to needs.

Indeed the goal of many scientist, biotechnologists, medical engineers, doctors, and surgeon requiring tools for dealing with infectious events is the ability to reduce working cycle, periodical maintenance, and replacements, and offer a more efficient treatment methodology thus able to remedy larger portions of the population in need, and to offer enhancment to currently available socio-economical performance. The method according to the present invation present competitive advantages, and important saving benefits especially for medical, biotechnological, hospitals, clinics, and agricultural, and industrial applications, thus enhancing the quality of life in the human sphare.

Further more, the methodology of the present invention disclose a real time treatment methodology by pulse power triggering of photochemical, and/or photocatalytical surface treatment processes, and further penetrative techniques (using surface treatment to trigger volume treatment). It is already known in theory that every material may oxidize or break down if energy is applied to it, having at least equivelant amount of energy as the one holding it's molecules and atoms together. The real world is very different—More specifically, due to already available vibrational excitation stages available to help break the “bond”, the methodology of the present invention disclosed several techniques the present invention offer the ability to keep operating equipment use in medical procedures safe at all times, offering much quicker turn over, shorter working cycle, and the ability to ensure high level of biocompatibility, acustic excellence, while turning noxious species on the surface of said medical instrumentation into more inocious, more managable forms in real time, especially beneficial for ultrasound procedures using photocatalytic water based silicon coupling gell, and for the protection of wide variety of medical instrumentation, and equipment peripherals. Most medical instrumentation are in need of sterilization, or disinfection leaving bacteria, and/or noxious species at a sufficiently low concentration according to standards, and health, and safety regulations. Further more, medical instrumentation used in wide variety of medical procedures are currently being treated with chemical disinfectants. As heat already being considered as one of the most expensive disinfectant, or sterilizing methodology often requiring long, and wastful work cycles schedule. More aspecifically, the long time cycle chemical disinfectant takes for effective inactivation of DNA & RNA replication sequences, or for oxidizing thus inactivating noxious species fule the drive for new more efficient, non chemical methodologies. Such is the method of the present invention, it is a non chemical, non residual treatment technology. The Present invention by using a photocatalytic compound made out of water, silicon, and photocatalytic material, such as TIO2,for example, the present invention disclose a novel methodology for treating wide variety of surfaces (of medical instrumentation) in short time facilitating the formation of workstations, and devices using the method of the present invention transffering pulsed laser light to a remote receptive interface, activating photocatalytic agent therein (in the coupling solution used). More specifically, the methodology of the present invention is using Ultraviolet light from about 200 nm, to about 400 nm to activate the photocatalytic agent present in the water based coupling gell (normally made out of silicon/water solution). By exposing the thin film coating left on probes, and ultrasound accessories, or body parts externally wounded, or for cuts, to a plureality of laser pulses in the region of the UVA, UVB, UVC, the present invention, triggers said thin film catalytically, and thus photocatalytically inucing beneficial photochemical processes (for example disinfection, and sterilazation), thus purifying, disinfecting, and inactivating noxious species on the plurality of surface described above (i.e. such as the surface of the medical instrumentations, wounds, cuts, sores, or externally damaged body surfaces). Further more, according to the method of the present invention said laser pulses having germicidal wavelength, and sufficient e/V energy for the specific treatment require for a particilar application wherein mild dissociation effects are beneficial for the methodology according to the present invention to offer photocatalytic solution in the field, or in disaster areas, or in places wherein the population is exposed to unexpected conditions dictating medical procedures to be performed without the infrastructure normaly associated with hardware equipment for disinfection (i. Such as aotoclave, ovens, Gamma rays, Radio waves, X-rays, microwaves, heat, cold, Sonication).

The present invention disclosed a novel methodology for photoreactive disinfection of surfaces, beneficial for the disinfection, and treatment of wide varieties of medical instrumentation, and for the thin-film coating, and activation on the surface of wounds, cuts, bruises, sores, damaged parts of external body surface), and in air, such as when infectious events may pause threat to humans, animals, and plants. Further more, by integrating into high purity water enriched with oxygen, and mixed with catalytic powder, or liquid, or gas, or light the methodology of the present invenvention facilitate the formation of active free radical layer swiftly making the surface curvature/proximities sterilized. Noxious species on the said surface are then inactivated, thus the surface is been made safe from infectioius events, (bacteria, viruses, and other health threatening noxious species). The present invention is extremely beneficial for many medical application hence there are no need for actual physical touching, terring, or swiveling of tisues, or there is no need for physically interfering with an already sensative, and often critical medical treatment scenarios. The present invention facilitate the formation of a free sterilized zone extending to reach the entirety of the spread of siad catalytic compound, naturally in line with environmental conditions such as wind, air compound, PH levels, dissolved Oxygen and othe factors effecting photocatalysis quantum yield of efficiency (100-107), (a-z), (a/1-z/10). (FIGS. 1-30).

Disinfection, purification, and sterilization and photo-treatment by Ultra Violet light technology is well known, this technology is preferred due to its non residual, non chemical, and effective (wavelength range from about 220 nm-357 nm) in inactivating DNA & RNA replication sequences in wide variety of noxious species, bacteria, viruses, Cysts, and Pathogens. Currently used methodologies utilizing UV light for disinfection, and photo-treatment is making use of (CW) Continuos Wave, often polychromatic light sources, most of which having radial emission, and not sufficient peak power (i.e. such as generated by P.W. type light sources). More specifically, the principle means for the generation of Ultra Violet light for disinfection and for photo-treatment is by using Mercury type light sources, or lamps. These lamps generate continues type of light (i.e. CW), and the majority of the light they generate (their peak emission) (mercury), is at the region of about 254 nm. These light sources/lamps, thus does not have the required wavelength for offering efficient disinfection, and sterilization of wide varieties of medical instrumentation. More specifically, current methodologies for disinfection, and sterilization of medical instrumentation include heat, Gamma rays, X-rays, Y-rays, radion waves, Ultra Violet, microwaves, chemicals. These methodologies while offering currently implemented solutions, imposed strict limitations on

ablation in the context of the present invention means the removal of material or tissue by melting, evaporation, or vaporization.

absorb in the context of the present invention means to transform radiant energy into a different form, usually with a resultant rise in temperature.

absorbance in the context of the present invention means the ability of a medium to absorb radiation depending on temperature and wavelength expressed as the negative common logarithm of the transmittance.

absorption coefficient in the context of the present invention means the amount of radiant energy absorbed per unit or path-length.

active medium in the context of the present invention means a medium in which lasing will take place, rather than absorption, at a given wavelength.

afocal in the context of the present invention means literally, “without a focal length”; an optical system with its object and image point at infinity.

air-cooled laser in the context of the present invention means a laser using fans to force air over the laser tube and through the power supply, air-cooled lasers have the benefit of needing no water supply, although the fan noise can sometimes be a disadvantage, usually only small and medium power lasers are air-cooled, very small

lasers, typically helium-neon, need no fans, although technically they are “air cooled” via convection, the term is usually applied only to fan-forced cooling.

amplification in the context of the present invention means the growth of the radiation field in the laser resonator cavity, as the light wave bounces back and forth between the cavity mirrors, it is amp stimulated emission on each pass through the active medium.

amplitude in the context of the present invention means the maximum value of the electromagnetic wave, measured from the mean to the extreme; put simply, the height of the wave.

angstrom unit in the context of the present invention means a unit of measurement for a wavelength of light (written å), equal to one ten billionth of a meter (10⁻¹⁰ meter). occasionally still used.

anode in the context of the present invention means an electrical element in laser excitation which attracts electrons from a cathode, an anode can be cooled directly by water or by radiation.

ar coatings in the context of the present invention means anti-reflection coatings, used on the backs of laser output mirrors to suppress unwanted multiple reflections which reduce power.

argon laser in the context of the present invention means a laser filled with argon gas, it gives off green and blue light, the strongest lines are at 514 nm (green) and 488 nm(blue), argons range from small 15 milliwatt 110 volt air-cooled models to large 50 watt 440 volt water-cooled systems, argon lasers are the most common type of light show lasers since they provide unable brightness at a reasonable cost.

average power in the context of the present invention means the sums of the energy of all single discrete pulses, in one second, of a pulsed laser.

autocollimator in the context of the present invention means a single instrument combining the functions of a telescope and a collimator to detect small angular displacements of a mirror by means of its own collimated light.

axial-flow laser in the context of the present invention means the simplest and most efficient of the gas lasers, an axial flow of gas is maintained through the tube to replace those gas molecules depleted by the electrical discharge used to excite the gas molecules to the lasing state.

axis, optical axis in the context of the present invention means the optical center-line for a lens system; the line passing through the centers of curvature of the optical surfaces of a lens.

beam diameter in the context of the present invention means the diameter of that portion of the beam which contains 86% of the output power.

beam expander in the context of the present invention means optical device increasing beam diameter and reducing divergence.

beam splitting in the context of the present invention means optically splitting a laser beam into two or more beams, of various or identical energys.

brewster windows in the context of the present invention means the transmissive end (or both ends) of the laser tube, made of transparent optical material and set at brewster's angle in gas lasers to achieve zero reflective loss of vertically polarized light non-standard on industrial lasers, but a must if polarization is desired.

brightness in the context of the present invention means the visual sensation of the luminous power of a light beam, as opposed to scientifically measured power of the beam.

calorimeter in the context of the present invention means an instrument which measures the heat generated by absorption of the laser beam—another way to measure laser power.

cathode in the context of the present invention means the element providing the electrons for the electrical discharge used to excite the lasing medium.

co₂ laser in the context of the present invention means a laser largely used in industry in which the primary lasing medium is carbon dioxide.

coaxial gas in the context of the present invention means most laser welding is done with a shield of inert gas flowing over the work surface to prevent plasma oxidation and absorption, to blow away debris, and to control heat reaction, the gas jet has the same axis as the beam so the two can be aimed together.

coherent light, coherent radiation in the context of the present invention means radiation composed of wave trains vibrating in phase with each other, coherent light waves all travel the same direction (spatial coherence) at the same frequency and in phase (temporal coherence), a laser produces coherent light, conventional light sources produce incoherent light.

collimated light in the context of the present invention means beam light rays traveling parallel to each other.

collimation in the context of the present invention means the process by which divergent rays are converted into parallel rays.

convergence in the context of the present invention means the bending of light rays toward each other, as by a positive (convex) lens.

current saturation in the context of the present invention means maximum flow of electric force in a conductor; in a laser, the point at which further electrical charge will not increase lasing action.

cw in the context of the present invention means an abbreviation of continuous wave of a laser as opposed to pulsed operation.

depth of field in the context of the present invention means the working range of the beam, a function of wavelength, diameter of the unfocused beam, and focal length of the lens, to achieve a small diameter spot size, and thus a high power density, a short depth of field must be accepted.

dichroic filters and mirrors in the context of the present invention means a piece of glass with an optical thin-film coating that transmits certain colours (wavelengths), and reflects the remaining colours, dichroic filters are used to combine or eliminate specific colours as needed in a laser projector, dichroic mirrors are used to maximise the amount of light reflected from a laser of a particular wavelength, dichroics should be handled with care to prevent damage to the coating, [see also: colour box]

diode laser in the context of the present invention means a semiconductor similar to an led (light-emitting diode) but which produces coherent light, diode lasers are small and efficient, which has led to their use in compact disc players and pen-type laser pointers, currently, diode lasers are too dim or expensive for most light show uses. this is likely to change over the next few years, [see also: solid-state laser]

divergence in the context of the present invention means the angle at which the laser beam spreads in the far field; the bending of rays away from each other, as by a concave lens or convex mirror.

drift, angular in the context of the present invention means all undesirable variations in output (either amplitude or frequency); angular drift of the beam, measured in milliradians before, during, and after warm-up.

duty cycle in the context of the present invention means the length of time the laser beam is actually cutting, drilling, welding, or heat-treating, as compared to the entire work cycle time.

electromagnetic wave in the context of the present invention means a disturbance which propagates outward from an electric charge which oscillates or is accelerated. includes radio waves; x-rays; gamma rays; and infrared, ultraviolet, and visible light.

emissivity, emittance in the context of the present invention means rate at which emission takes place; the ratio of the radiant energy emitted by a source or surface to that emitted by a blackbody at the same temperature.

exposure in the context of the present invention means a measure of the total radiant energy incident on a surface per unit area; radiant exposure.

far-field imaging in the context of the present invention means an imaging technique with solid-state lasers that has several limitations: non-uniform energy distribution, very short working distances, and poor control of hole geometry.

fiber optic cable in the context of the present invention means flexible glass or plastic strands made into a cable, used to carry light from one place to another, there are two main types; step index and graded index fiber, within these two main types there are two further subgroups:

transmission fibers carry the beam with as little loss as possible, they are used to transmit the laser's light to remotely located projection devices.

display fibers [also knows as side glow fibers] have no cable jacket, so some light scatters out the side of the strands, the strands themselves look like microscopic neon tubing and become a special effect, such as a laser-lit “whip” or a glowing “rope” wrapped around objects.

flashlamp in the context of the present invention means source of powerful light; often in the form of a helical coil and used to excite photon emission in a solid-state laser.

fluorescence in the context of the present invention means the glow induced in a material when bombarded by light, brewster windows of fused silica fluoresce in uv light, increasing absorption of laser radiation and degrading laser mode and output.

flux in the context of the present invention means the radiant, or luminous, power of a light beam; the time rate of the flow of radiant energy across a given surface.

focus in the context of the present invention means noun: the point where rays of light meet which have been converged by a lens, verb: to adjust focal length for the clearest image.

focal point in the context of the present invention means (same as first definition under “focus;” in laser work,) the focal point of the beam relative to the work surface has a critical effect, such as the depth and shape of drilled holes, when the focal point is at the surface, holes are of uniform diameter, when the focus is below the surface, conical holes are drilled.

folded resonator in the context of the present invention means construction in which the interior optical path is bent by mirrors mounted on corner blocks bolted into pre-aligned position, permitting compact packaging of a long laser cavity.

frequency in the context of the present invention means the number of light waves passing a fixed point unit of time, or the number of complete vibrations in that period of time.

gain in the context of the present invention means another term for amplification, usually referring to the efficiency of a lasing medium in attaining a population inversion, high gain is typically more than 50% per pass of the light wave between cavity mirrors.

gas discharge laser in the context of the present invention means a laser containing a gaseous lasing medium in a glass tube in which a constant flow of gas replenishes the molecules depleted by the electricity or chemicals used for excitation, the discharged gas can be filtered and 90% recycled for economy.

gas jet assist in the context of the present invention means an assisting coaxial gas, such as oxygen, argon, or nitrogen, which may be used to achieve very high power levels for cutting certain metals.

gas transport in the context of the present invention means a laser design, which generates very high beam power within a fairly small resonator structure, long electrodes parallel the axis and gas is circulated across the resonator cavity.

gaussian in the context of the present invention means the “normal curve,” or normal distribution, an example of which is the symmetrical bell shape of the holes created by the uncorrected, unfocused laser beam in its optimum mode, a gaussian laser beam has most of its energy in the center.

haz in the context of the present invention means heat-affected zone, or the area where laser beam and metal (or other) surface are in contact.

helium-neon laser in the context of the present invention means (“hene”), laser in which the active medium is a mixture of helium and neon, which is in the visible range, used widely in industry for alignment, recording, printing, and measuring, it is also valuable as a pointer or aligner of invisible co₂ laser light.

heat sink in the context of the present invention means a substance or device used to dissipate or absorb unwanted heat, as from a manufacturing process (or, with lasers, from reflected rays).

hertz in the context of the present invention means the approved international term, abbreviated hz, which replaces cps for cycles per second.

image in the context of the present invention means the optical reproduction of an object, produced by a lens or mirror, a typical positive lens converges rays to form a “real” image which can be photographed, a negative lens spreads rays to form a “virtual” image which can't be projected.

incident light in the context of the present invention means a ray of light that falls on the surface of a lens—or any other object, the “angle of incidence” is the angle made by the ray with a perpendicular to the surface.

intensity in the context of the present invention means the magnitude of radiant energy (light) per unit, such as time or reflecting surface.

ion laser in the context of the present invention means a type of laser employing a very high discharge current, passing down a small bore to ionize a noble gas such as argon or krypton, the ionization process creates a population inversion for lasing to occur, a research laser useful for some industrial applications.

ionization in the context of the present invention means the process by which ions are formed.

irradiation in the context of the present invention means exposure to radiant energy, such as heat, x-rays, or light; the product of irradiance and time.

joule in the context of the present invention means one watt per second; a measurement frequently given for laser output in pulsed operation.

krypton laser in the context of the present invention means a laser filled primarily with krypton gas, when used with “all-line” or “white” optics, it produces red, yellow, green and blue light, a “red-only” krypton laser uses with specially tailored optics to output a very strong red line at 647 nm, kryptons are similar to argons (the same tube design can be used for both), however, krypton gas produce less light (output power) than an equivalent volume of argon gas.

krypton lasers are primarily used when a powerful red light is needed.

laser in the context of the present invention means “laser” is an acronym derived from “light amplification by stimulated emission of radiation”.

a device which produces a coherent beam of light, the beam remains parallel for long distances and contains one or more extremely pure colours, light show lasers are usually gas-filled tubes using high voltage current to ionise the gas (cause the gas to glow), mirrors at each end of the tube help amplify a process called “stimulated emission”, most of the stimulated emission light travels between the two mirrors; between 1% and 4% comes out of one of the mirrors to create the beam of light that we see, the gas used determines the colour (or colours) of the beam, gas lasers remain the overwhelming choice for display applications, the four main types used are a helium-neon mixture, argon, krypton, and an argon-krypton “mixed gas” mixture.

laser oscillation in the context of the present invention means the buildup of the coherent wave between laser cavity end mirrors, in cw mode, the wave bounding back and forth between mirrors transmits a fraction of its energy on each trip; in pulsed operation, emission happens instantaneously.

laser rod in the context of the present invention means a solid-state, rod-shaped lasing medium in which ion excitation is caused by a source of intense light, such as a flashlamp, various materials are used for the rod, the earliest of which was synthetic ruby crystal.

light in the context of the present invention means the range of visable electromagnetic radiation frequencies detected by the eye, or the wavelength range from about 400 to 750 nanometers, it is sometimes extended to include photovoltaic effects and radiation beyond visible limits.

light regulation in the context of the present invention means a form of power regulation in which output power is maintained at a constant level by controlling discharge current.

luminance in the context of the present invention means commonly called illumination; the luminous or visible flux per unit area on a receiving surface at any given point.

meniscus lens in the context of the present invention means the lens used primarily in co₂ lasers by coherent, inc. it has one side convex, the other concave.

metastable, metastable state in the context of the present invention means unstable condition in which the energy of a molecule is at some discrete level above the lowest, or ground state, it is this condition which is necessary for emission of photons in a laser. (from quantum theory.)

millijoule: one thousandth of a joule.

milliwatt in the context of the present invention means one thousand milliwatts equal one watt, small lasers' beam powers are measured in milliwatts, for example, a 50 mw laser is one-twentieth of a watt; a 500 mw laser is one-half watt.

mode in the context of the present invention means a particular functioning arrangement, setup, or condition for laser operation, such as continuous emission, pulses, or grouped pulses. “mode” also describes the cross-sectional shape of the beam. (see “tem.”)

modulation in the context of the present invention means the ability to superimpose an external signal on the output beam of the laser as a control.

monochromatic light in the context of the present invention means theoretically, light consisting of just one wavelength, since no light is completely monochromatic, it usually consists of a very narrow band of wavelengths, lasers provide the narrowest bands.

nanometer in the context of the present invention means a unit of length in the international system of units (si) equal to one billionth of a meter (10⁻⁹ meter), once called a millimicron, it is used to represent wavelength, abbreviated “nm.”

near field imaging in the context of the present invention means a solid-state laser imaging technique offering control of spot size and hole geometry, adjustable working distance, uniform energy distribution, and easily produced range of spot sizes.

nd:glass laser in the context of the present invention means a solid-state laser of neodymium:glass offering high power or short pulses, or both, for specific industrial applications.

nd:yag laser in the context of the present invention means a solid-state laser of neodymium:yttrium-aluminum garnet, similar to the nd:glass laser, both are pumped by flash lamps, or diode lasers.

nema in the context of the present invention means national electrical manufacturers' association, a group which defines and recommends safety standards for electrical equipment.

noise in the context of the present invention means unwanted, minor currents or voltages in an electrical system.

object in the context of the present invention means the subject matter or figure imaged by, or seen through, an optical system.

optical density in the context of the present invention means protection factor provided by a filter (such as used in eyewear, viewing windows, etc.) at a specific wavelength, each unit of od represents a 10× increase in protection.

optical pumping in the context of the present invention means exciting the lasing medium by the application of light rather than electrical discharge from anode and cathode.

output coupler in the context of the present invention means the resonator mirror which transmits light; the one at the opposite end is totally reflective.

output power in the context of the present invention means the energy per second emitted from the laser in the form of coherent light, usually measured in watts for continuous-wave operation and joules for pulsed operation.

peek power in the context of the present invention means the power of an individual pulse in a pulsed laser, it is obtained by dividing the pulse energy in joules to the pulse width in seconds, typical values can be reach mega and giga watts.

photoacoustic effects in the context of the present invention means arises with the use of very short-duration high-energy laser pulses, at pulse durations typically below 10 microseconds, significant amounts of energy are absorbed and a rapid expansion occurs in the tissue, generating an acoustic shock wave that causes mechanical disruption to cellular structures.

photochemical effects in the context of the present invention means effects that occur from long exposure durations at incident power levels insufficient to cause damaging photothermal effects, it is an energy dependent process (a function of the total quantity of radiation absorbed rather than its rate of absorption).

photometer in the context of the present invention means an instrument which measures luminous intensity.

photon in the context of the present invention means in quantum theory, the elemental unit of light, having both wave and particle behavior, it has motion, but no mass or charge.

photothermal effects in the context of the present invention means the damage mechanism for acute laser injury (i.e. for injury immediately following exposure), the radiation incident at the surface is absorbed in the underlying tissue, increasing the temperature of the tissue to the level at which damage can occur, and laser burns result, it is a power dependent process (a function of the rate at which energy is absorbed rather than the total quantity of energy involved).

plasma in the context of the present invention means in laser welding, a metal vapor that forms above the spot where the beam reacts with the metal surface, also used to describe the laser tube (plasma tube, discharge tube) which contains the completely ionized gas in certain lasers.

polarization in the context of the present invention means restriction of the vibrations of the electromagnetic field to a single plane, rather than the innumerable planes rotating about the vector axis, this prevents optical losses at interfaces between the lasing medium and optical elements, various forms of polarization include random, linear (plane), vertical, horizontal, elliptical, and circular, of two polarization components (so-called), s and p, the p component has zero losses at brewster's angle.

population inversion in the context of the present invention means when more molecules (atoms, ions) in a laser are in a metastable state than in the ground state (a situation needed for sustaining a high rate of stimulated emissions), a “population inversion” is said to exist, without a population inversion, there can be no lasing action.

power density: the amount of radiant power concentrated on a surface, units watts per square meters or square centimeters

pulse energy in the context of the present invention means the energy of a single, brief emission from a laser programmed for pulsed behavior rather than continuous operation, pulse power can be several times greater than cw emission.

pulse tail in the context of the present invention means pulse decay time, which can be shortened (by using a special mixture of gases) to allow for fast repetition of laser pulses within a given length of time.

q-switch in the context of the present invention means a device that has the effect of a shutter moving rapidly in and out of the beam to “spoil” the resonator's normal q, keeping it low to prevent lasing action until a high level of energy is stored, result: a giant pulse of power when normal q is restored.

quasi cw in the context of the present invention means the pulsating of a continious light into pulsed light by acusto optic, electrooptic, electronic, or mechano optical means, so peak powers are reduced, by number of pulses (see rep, rate) are increased.

radiance in the context of the present invention means brightness; the radiant energy per unit solid angle and per unit projected area of a radiating surface.

radiant energy in the context of the present invention means energy traveling as wave motion; specifically, the energy of electromagnetic waves (light, x-rays, radio, gamma rays).

radiant flux: the rate of emission or transmission of radiant energy.

radiant intensity in the context of the present invention means radiant power, or flux, expressed as emission per unit solid angle about the direction of the light in a given length of time.

radiant power in the context of the present invention means the amount of radiant energy available per unit; the radiant flux.

reflectance in the context of the present invention means the ratio of the reflected flux to the incident flux, or the ratio of reflected light to light falling on the object.

reflection in the context of the present invention means the return of radiant energy (incident light) by a surface, with no change in wavelength.

refraction in the context of the present invention means the change of direction of propagation of any wave, such as an electromagnetic wave, when it passes from one medium to another in which the wave velocity is different, simply put, the bending of incident rays as they pass from one medium to another, such as air to water.

resolution in the context of the present invention means resolving power, or the quantitative measure of the ability of an optical instrument to produce separable images of different points on an object; the capability of making distinguishable the individual parts of an object, closely adjacent images, or sources of light.

resonator in the context of the present invention means the mirrors (or reflectors) making up the laser cavity containing the laser rod or tube, the mirrors reflect light back and forth to build up amplification under an external stimulus, emission is through one of them, called a coupler, which is partially transmissive.

rockwell c in the context of the present invention means a scale or test used to define hardness in metals, particularly steel and titanium.

solid-state laser in the context of the present invention means a laser where the lasing medium is a solid material such as a ruby rod, these can be optically pumped by a flashlamp or diodes, solid state lasers also include diode lasers as they use electrically pumped solids to produce light.currently, solid-state lasers are too expensive for most light show uses, this may change over the next few years, the most promising solid-state laser uses a material called nd:yag, which produces infrared light, this can be frequency doubled (second harmonic generation) to produce up to 60 watts of green light at 532 nm, the green light can again frequency doubled (fourth harmonic generation) to produce uv light at 266 nm, up to several watts.

spectral response in the context of the present invention means the response of a device or material to monochromatic light as a function of wavelength.

stimulated emission in the context of the present invention means when an atom, ion, or molecule capable of lasing is excited to a higher energy level by an electric charge or other means, it will spontaneously emit a photon as it decays to the normal ground state, if that photon passes near another atom of the same frequency which is also at some metastable energy level, the second atom will be stimulated to emit a photon. both photons will be of the same wavelength, phase, and spatial coherence, light amplified in this manner is intense, coherent (collimated or parallel), and monochromatic, in short, laser light.

tem in the context of the present invention means abbreviation for transverse electromagnetic mode, the cross-sectional shape of the working laser beam, an infinite number of shapes can be produced, but only a relatively small number are needed for industrial applications, in general, “the higher the tem, the coarser the focusing.”

tem00: a gaussian-curve mode that is the best collimated and produces the smallest spot of high power density for drilling, welding and cutting.

tem01: divided into two equal beams for special applications.

threshold in the context of the present invention means during excitation of the laser medium, this is the point where lasing begins.

transmission in the context of the present invention means in optics, the passage of radiant energy (light) through a medium.

transmittance in the context of the present invention means the ratio of transmitted radiant energy to incident radiant energy, or the fraction of light that passes through a medium.

vignetting in the context of the present invention means the loss of light through an optical element when the entire bundle does not pass through; an image or picture that shades off gradually into the background.

visible light transmission/transmittance in the context of the present invention means the amount of visible light usable to the eye that passes through a filter, as a rule of thumb, as optical density increases, visible light transmission decreases—but not always.

watt in the context of the present invention means an objective measure of power; in lasers, usually refers to the optical output power, or strength, of a laser beam, watts are also used in a more conventional sense, to measure electrical power used by a laser, for example, a 10 w (optical) argon laser consumes around 10,000 w of electrical power.

wave in the context of the present invention means an undulation or vibration, a form of movement by which all radiant energy of the electromagnetic spectrum is thought to travel.

wavelength in the context of the present invention means the fundamental property of light—the length of the light wave, which determines its color, common units of measurement (which is usually from crest to crest) are the micron, the nanometer, and (earlier) the angstrom, visible light has wavelengths extending from about 700 nanometers (red) through orange (˜600 nm), yellow (˜580 nm), green (˜550 nm), blue (˜450 nm) and violet (˜400 nm).

white-light beam in the context of the present invention means broadly, a laser beam which contains a number of different wavelengths (colours) so the beam appears white, if the beam is passed through a prism or diffraction grating, it is separated into

individual laser beams, each of a single specific wavelength, more specifically, a white-light beam ideally contains twice as much red as green and blue light for correct colour balance (see appendix), it can be from a single white-light laser or from two or three lasers whose beams have been combined into a single beam, white-light beams are primarily used in rgb laser projectors.see the definition of white-light laser for more information on what constitutes an “equal mixture” of light

white-light laser In the context of the present invention means Many lasers can produce a number of wavelengths (colours) simultaneously. A white-light laser is designed to give a good balance of red, green and blue wavelengths. Usually the laser is intended for an RGB laser projector. (Some models also deliberately add yellow light for specialised 4-colour projectors.) Most white-light lasers use an anrgon/krypton gas mixture. It is somewhat

difficult to produce an equal balance of desired colours, and to keep this balance consistent during the lifetime of the laser tube. At present, there are no standards defining the exact wavelengths and colour proportions for a laser to be called

“white-light”. In addition, the sought-after colour balance can be defined either as equal amounts on a photometer, or as visually equal amounts. Since the eye is much more sensitive to green, a visually equal or “photopically balanced” laser has roughly five times more power in red and blue than in green. Most white-light lasers today are not photopically balanced.

window In the context of the present invention means A piece of glass with plane parallel sides which admits light into or through an optical system and excludes dirt and moisture.

A further environmental embodiment of the method of the present invention wherein the content of the bottles, conduits, or chambers may be selected from: beverages, wine, medical preparation, juice, drinking water, mineral water, insulin products or medical preperation. Spring water, flavored water, flavored beverages, biological traceable compounds, Drug delivery using water based, and/or expanded or flavored water drinks containing vitamines or nutrients, alcohol, blood products, plasma products, air products, gases for propelling medications, sprays, or any liquid or gasses or hybrid combination thereof.

A Novel environmental embodiment of the present Invention is having a high repetition rate, high peak power laser of the Nd:Yag, or Nd:Glass, or Nd: YLF, type or any combination thereof operating in the Fourth Harmonic generation mode (i.e. FHG), a further preferred embodiment of the present invention is having said solid state (i.e. Nd: Yag type for example), working in the Third Harmonic Generation mode (i.e. THG), a preferred embodiment according to the method of the present invention is having an electrical discharge laser such as an excimer laser operating in wavelength from about 193 nm, through to about the 308, and 351 nm, and wherein each of said pulses of light is aligned

into the content of the bottled liquids or gasses for purifying, disinfecting, and ensuring that DNA, and RNA replication sequences are thus inactivated, providing a non invasive disinfecting methodology wherein light pulses from the laser are penetrating the material from which the bottles, (i.e. conduits or chambers, or bottles, or pipes) are made.

A preferred embodiment according to the method of the present Invention is having a hybrid laser system operating wherein (a) a solid state laser operating at THG (355 nm), is attached, or aligned to the transparent lid, or cork, or encapsulation, or conduit, or chamber, and wherein together in hybrid format an electrical discharge laser is also operating for maximizing the efficiencies associated with the non destructive disinfection according to the method of the present invention. Further prefered embodiment according to the present invention wherein the packaging is a liquid waveguide, or concventrator.

A preffered embodiment of the present invention wherein a Nd:Yag laser is used having a third Harmonic generator producing 355 nm wavelength which pentrates the surface of the skin in human, and animals and thus swiftly inactivates DNA & RNA replication sequences in blood therein (in the blood, i.e. under the skin, non invasively). Such preffered embodiment of the present invention could be implemented with at least one laser unit having its beam of pulsed UVA light split, and distributed to a plurality of location by means of waveguiding light through HGFS optical fibers, photonic band gap waveguides, or polymer waveguides, or hybrid combinations, each distal tip is attached, supported, or threaded, in proximity or distributed to cover areas inthe body wherein blood vessels are relatively exposed, and are conviniently positioned around the surface (i.e. inporoving transmission, and coupling conditions). A further preffered embodiment of the present invention is wherein several lasers are used having high repetition rates, or high peak powers, and pulse duration from about 1 ms, to about 1 fs, or Atosecond and wherein their respective beams are collected, or diflected, or diverted or sttired to form a three dimension element of UVA light at high energy density, and wherein said high energy density is below the damage threshold of blood components, nutrients, conditioners, complimental systems, MHC type 1,2,3, and any vital cell present in blood or blood products, yet they are efficient for inactivation of noxious species therein, i.e. within the blood, or blood products.

A preffered embodiment of the present invention wherein the laser light source is sellcted from (a) Gas discherge laser, (b) diod pumped lasers, (c) plasma discharged lasers, (d) solid state lasers, (e) semi conductor lasers, (f) crystal type of lasers, (g), X-rays pumped lasers, (h) E-beam pumped gas lasers types,

(I) FEL (Free Electron Laser amplifier), (j) EA/FEL (Electrostatically Accelerated Free Electron Laser), or organic laser types or any combination thereof.

A preffered embodiment of the present invention wherein the laser light source is tunable from about 1 nm to about 3000 nm, a further preffered embodiment of the pesent invention is wherein the laser light source is tunable from about 333 nm to about 360 nm, and wherein the peak power density of individual pulses reach from about 1 nJ/Cm², to about 50 Js/Cm², and wherein said pulsed laser light source is pulsing at repetition rates from about 1 Hz to about 300 MHz thus the preffered embodiment of the present invention is suitable for wide ranging application involving different packaging materials, thus their specie specific optical caliberation standards are calculated for a specific biodosimetric value or curve to appropriately corasponde (being lower) with damage threshold of the substrate material used in a specific application, or tool, or device.

### DETAILED DESCRIPTION OF THE FIGURES

The figures according to the method of the present invention illustrate preferred embodiments of the present invention, and block schematics of devices using the methodology of the present invention, and as such, do not intend to limit the scope of the present invention what so ever; FIGS. 1-10, are herewith disclosed, and forthwith are herewith presented for clarity illustrating competitive advantages, benefits, and modularity in which scaling down or up design criteria for devices according to the method of the present invention mat be performd, and excecuted.

The figures according to the method of the present invention illustrate preferred embodiment of the present invention do not intend to limit the scope of the present invention what so ever; FIGS. 1-10, together with scientific support data is herewith disclosed:

FIG. 1 illustrates a block schematic of the present invention for sterilization surfaces of a vehicle or car according to the methodology of the present invention. A car (102) is shown being disinfected by a mobile photo-catalytic (hand held) device (106, 105, 107) held by a person (100) standing in proximity to said car. An optical fiber (107) is illustrated for clarity connected at one end to a pulsed UV laser (not shown), and at the other end to the catalytic hand held device (106), light from the laser is delivered through the optical fiber to the hand held device and being projected (105, 105a, 105b) to the surface of the contaminated (105c) car surface being sterilized. Advance oxidation processes are being initiated by the photochemical interaction of the laser with the oxidizer (105a)shown in droplet format (105a), especially beneficial for protection against contaminants of biological, and/or chemical origin, and for photo-treatment, and disinfection of wide variety of surface in metropolitan areas, and in domestic, commercial or medical applications.

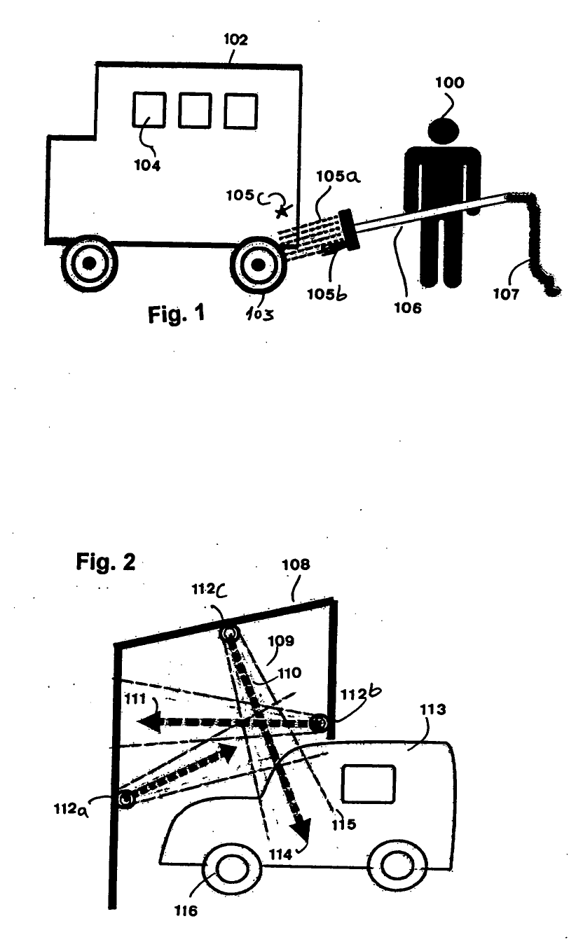

FIG. 2 illustrates a block schematic of the present invention for sterilization surfaces of a vehicle or car automatically according to the methodology of the present invention. (108) represent support means of the laser (not shown), and for the oxidizer jets (112a, 112b, 112c), a car is illustrated (113), and the light (114, 111, 109, 110) is shown to interact with the oxidizer suspension or H2O2 water based compound sprayed (115), radical species (OH—, not shown) are thus produced according to the methodology of the present invention triggered by the pulsed laser light (114), the car is static or moving through the automatic car sterilization support means (108) for advanced oxidation processes to be initiated by the laser light interacting with the oxidizer (water based H2O2 suspension).

FIG. 3 illustrates a schematic view of the present invention for sterilization of air volume and/or surfaces in metropolitan areas according to the methodology of the present invention. (117) illustrate a person holding a portable pulsed UV laser (118) device using the methodology of the present invention. The beam of the portable laser is illustrated in (119), the pulsed UV laser beam (119, 127) is shown traversing photo-catalytic bubbles which have being released or delivered, or produced in situ (not shown) at the contaminated areas. (120) represent a bubble hit by the laser creating an acusto-optic effects, diffusing, and scattering the light from the pulsed UV laser in 360 degrees, such that it could trigger bubbles in the proximity of the 1st effected bubble, a spore or contaminated specie (124) is illustrated effected by the free radical species created by the interaction of the pulsed UV light (121, 1222, 123, 126, 120, ) from the laser (118, 119). An additional person is standing near by in proximity (129, 128) to illustrate the high safety levels of the methodology of the present invention, the physical distance between (124), (126) illustrate the capabilities of the methodology of the present invention wherein the pulsed laser light is the trigger or match, and the scattered light causing advance oxidation processes to occur is the fire, thus the size of the match does nope correspond with the size of the fire. The photo-catalytic bubbles are thus facilitating disinfecting, or sterilization or dissociation or oxidation processes to occur in places, locations wherein the actual light have nor reached or covered, substantially increasing the geometrical utilization of the methodology of the present invention. (1120, 125, 126, 127) illustrate the light scattered, and diffused from the original hit bubble (120), thus covering and treating areas larger then the areas covered by the light from the laser alone.

FIG. 4 illustrates a block schematic view of the present invention for sterilization of surfaces, and/or air volumes in a multi store/floor building according to the methodology of the present invention. (130) illustrate a central laser system coupled (not shown)(to a harness of optical fibers (131) 133,) to extending to reach individual trajection or projection points throughout the building floors (in each floor) (132, 135, 134,) light from the laser is interacting with the oxidizer photo-catalytic compound, or bubbles (147, 146, 144, 143, 140, 141, 138) for the purpose of producing free radical species (not shown). Much like the sprinklers gains fire which are installed in hotels, and other key building, the methodology of the present invention and devices for use thereof could be installed in a building's infrastructure support means (1336a,b,c,), (137, 154), (154) represent the ceiling or floor of the 1st floor, people are illustrated in each floor (150, 142, 139,) being protected by the method and related devices according to the present invention.

Advanced oxidation processes are occurring in each floor inactivating DNA replication sequences of noxious species, or dissociating and reducing toxic chemical species which may enter the building. (152a,b,c) represent nozzles for dispersing the catalytic compound in bubbles or vapor or droplet formats, H2O2 could be added to the general water utility (not shown) or additional oxygen could be dissolved into the water utility just before use, in situ, in real time, and at concentration according to the specific threat or eventuality. (155) illustrate the floor, or ground zero, (156) illustrate the roof top of the building.

FIG. 5 illustrates a schematic view of the method of the present invention wherein people (157a,b,c,d, 158, 159, ) contaminated with noxious or toxic chemical or biological components or multi components are being cleaned by a photo-catalytic mobile sterilization, and dissociation interface (160). Pulsed UV light from an attached laser (not shown) is arriving through optical fibers, or waveguides (165), connected at one end to the laser (not shown), and at the other end (164) connected to the extendable integrated arm (160), (162a) illustrate the catalytic oxidizer being dispersed, and distributed, extending to reach the contaminated skin, or cloth of the contaminated people, (162b) illustrate a portion of the beam of light together with the oxidizer compound thus creating free radical species and sterilizing, disinfecting, cleaning, and oxidizing, thus making noxious species inactivated, and much more innocuous, and manageable, minimizing damage, saving lives in threatening eventualities. (161) Represent a jet, or diffuser, dispersed interface, or projecting element telescopically adjusted so as to fit the appropriate surface curvature of the area to be treated. And so Advanced Oxidation processes are initiated, triggered by the pulsed UV laser for making safe wide variety of surfaces.

FIG. 6 illustrates a schematic view of devices using the methodology of the present invention.

Preferred mode of utilization using a photo-catalytic, reactive bubble (166) technology for treatment of surfaces, liquids, and gasses volumes. (167, 169c, 171, 170, 169b, 169a, 168, 169, d) illustrate reflected pulsed UV light from the laser or high intensity light source (not shown), the opto-acoustic vibration created at the bubble level affect light which transverse said bubble, and thus effect additional bubbles (not shown) which are in proximity, such that the energy threshold required for the triggering of AOT (Advanced Oxidation Processes) in the surrounding areas, and vicinity of the original bubble first interacted with the light, especially beneficial when scanning larger area, surfaces, air volumes. In this figure, the size of the match (i.e. the laser, not shown) does not correspond with the size of the fire (meaning the radical species OH* thus created). Especially beneficial for protection against chemical, or biological noxious or toxic species pausing health threatening eventualities.

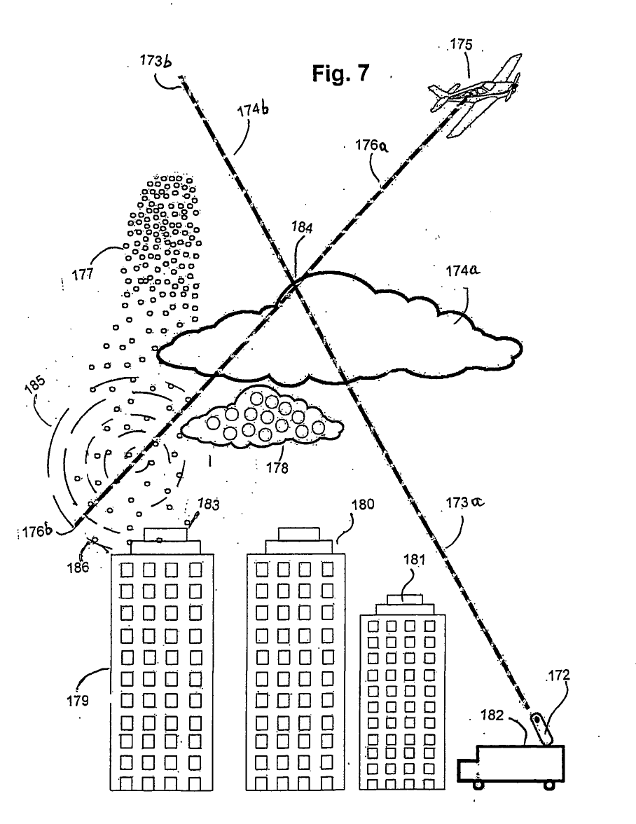

FIG. 7 illustrates the capabilities of the methodology of the present invention according to the methodology of the present invention for treatment of larger areas, surfaces, air volumes, and combination wherein (175) represent an air born pulsed UV laser, (182, 172) illustrate mobile ground based UV pulsed laser, light beams from the lasers are extending to reach the photo-catalytic, and photo-reactive oxidizer species (bubbles, or droplet format) represented by (177). (176) represent the light from the laser after passing through several bubbles or droplets and (185) illustrate the opto-acoustic effects and scattering, and reflecting, diffusing of the light (186), (178) represent toxic, or noxious species in need of immediate inactivation or reduction (biological or chemical species). (181, 180, 178, 183) illustrate building in a metropolitan area or city, or key building or installation (179) illustrate the height of the building relative to the surrounding areas, (174a) illustrate the remaining by products which are mainly CO2, and H2O which are harmless, and does not effect the environm ent leaving no toxic residuals. (182) represent the vehicle or track carrying the laser (mobile)(173) illustrate light passing from the ground upwards, (194) represent constructive interference created by the two or several laser beams interacting together.

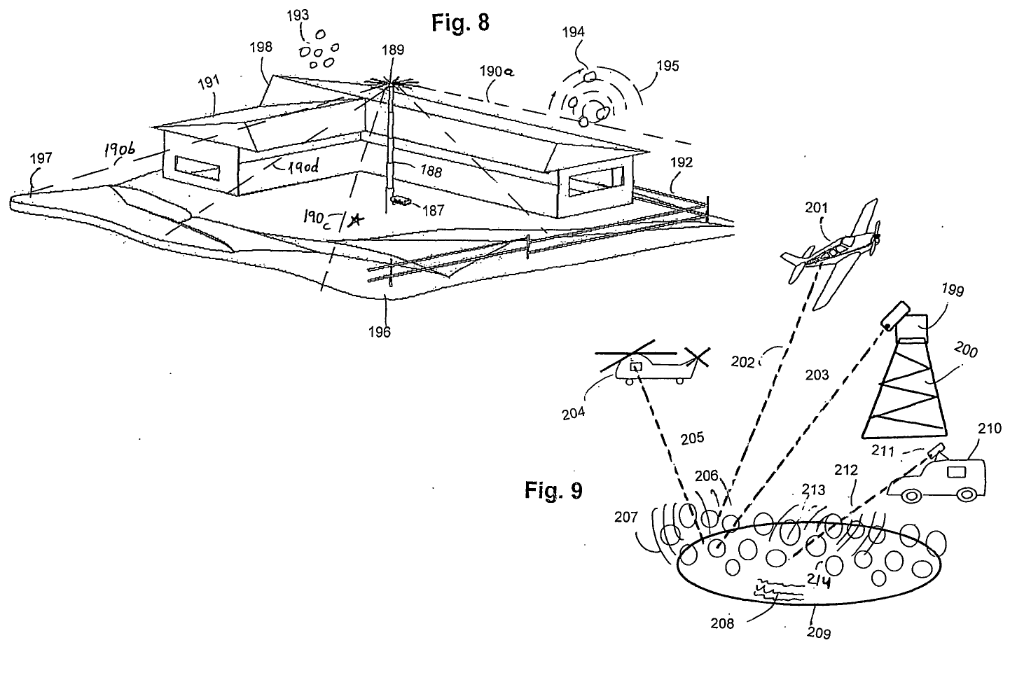

FIG. 8 illustrates schematic view of the devices using the method of te present invention for territorial protection of key building, farms, industrial or commercial sites, and predetermined areas. (189) Illustrate the light from the laser being applied over large area (scanning head), the laser is positioned on the ground (187), and its light is delivered through the central pole (188), (190a,b,cd) represent the portion of the laser beams covering different portion of the area to be protected/treated. (197) Illustrate the end point of the area to be protected, (191) illustrate a building in the area, (192) illustrate the other end of the defined protected area, ((190c) illustrate a toxic chemical specie or biological noxious specie which have entered the defined protected area and thus have being disinfected, sterilized, inactivated or dissociated or oxidized or mineralized or combination. (196) Illustrate the ground zero point of the ground, (193) represent additional toxic or noxious biological components or chemical substances which have not yet entered the field of action (i.e. the area protected by the device using the met9idilogy of the present invention. (195) Illustrate the acoustic-optic effects of the distributed, reflected, or scattered light from the bubbles as results of the interacting light from the laser.

FIG. 9 illustrates a schematic view of the methodology of the present invention for treatment of the water surface of lakes, rives, and water resources according to the present invention wherein (209) illustrate air-born laser, (199, 200) illustrate a ground based laser, (204) illustrate a helicopter (air born laser), the surface of the water is represented by (208), (209) illustrate the boundary of the water resource, (210) illustrate the vehicle carrying the mobile laser, (203) represent pulsed UV laser in the sub-microsecond time duration (per pulse), (214) illustrate the acoustic-optic effects helping to diffuse, and distribute the light to the surrounding area wherein additional catalytic compounds in forms of bubbles, droplets, or streams is illustrated (207, 214, 206, 207) the light is swiping or scanning the surface of the water wherein the dangerous mosquito such as the one promoting fever of the Nile, and other dangerous diseases, and thus enhancing the biosecurity of the area in proximity to the said water resource. (205, 206, 203, 212, 202) illustrate for clarity the pulsed UV laser light being applied by the methodology of the present invention.

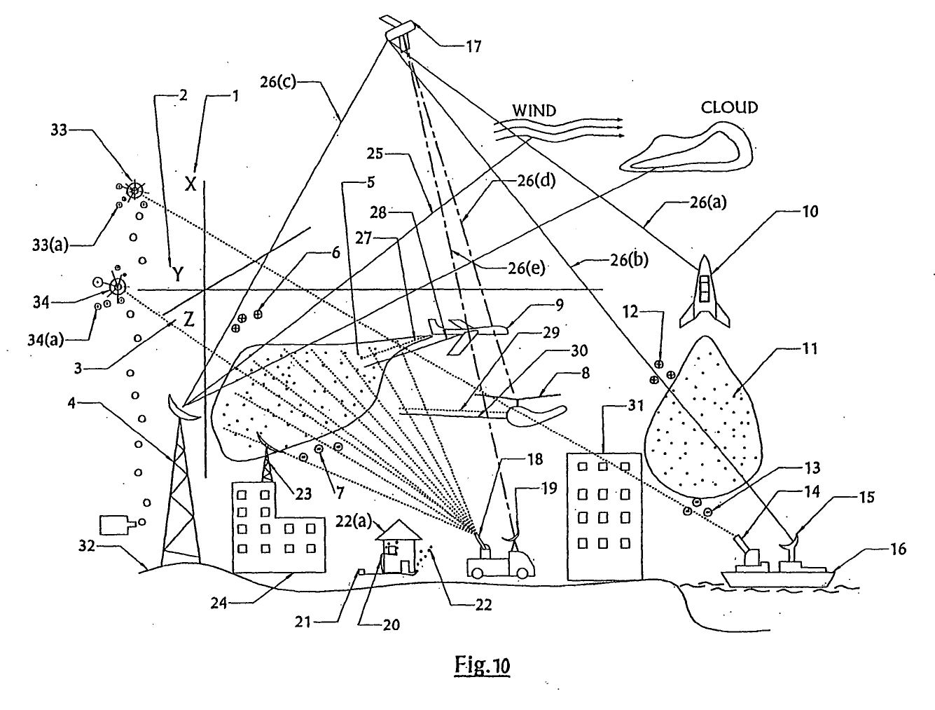

FIG. 10 illustrates a panoramic scale up view of globally positioned devices on land, air, and/or sea using the method of the present invention a schematic view for non interfering, non residual catalytic/sceniliting disinfection, and detoxification, sterilization and dissociation treatment to exact coordinates is herewith disclosed comprising:

(1), (2), (3), illustrate coordinates, relating to altitude, distances, hight, directional orientation, and targeting information, hence of the triggering laser beams, and hence of the diagnostic measurements relating to the data equasition involved in the spreading of the photocatalytic compound according to the present invention (1-34), (4) represent an antenna recieving ground truth monitoring information relating to weather information, and ground truth information relating to winds, clauds, smoke, catalytic dispersing areas, trigering efficiencies, spectral distribution measurements, and other sensing relating to the quality of air, liquids, and gasses, present, and atmospheric sensing of components which may effect the dosimetric valuers set for a predetermine d specific triggering event (illustrated here in air). (5) illustrate a claud type dispersion from passing air plane (9) of the photocatalytic scintillating comppound according to the methodology of the present invention, (6) illustrate noxious speicies introduced ion to the field of action (FIG. 1) by external source (not shawn), (7) represent inactivated species carrying the marking (−), in contradistinction to (6=+) shwn above the catalytic cloud dispersion; thus the processes of inactivation, dissociation, and advance oxydation is herewith disclosed and illustratedi a helicopter (8) shawn triggering, and sensing simultaniously, or sequnecially (Two primery beams29,&30), (9) illustrate a plane dispersing the catalytic compound according to the methodology of the present invention, the plave is also shawn projecting two beams one pulsed dotted line, one continious wave shawn for clarity in a continious line, (10) illustrate a rocket propelled vhiecle shawn having trhee squares in theside, these reprsent recieving data from sattalite (17), and from ground truth monitoring station (16, 19, 4, 23). (11) illustrate the catalytic compound of this invention discharged at a predetermined hight (x/y/z) altitude or speed (12) represent live noxious species, potent, wherein (13) illustrate inactivated, disinfected, catalytically oxydized species marked with negative sign (−). (14) represent the laser gun stationed on a ship (16), (15) illustrate radar antenna recieving the satlite communication (17), 16 illustrate the ship itself, (17) illustrate th4e sattalite giving cordination, (18) laser gun stationed on the mobile track, (19) represent antenna to recieve GPS positioning data, (20) illustrate dripping pipe designated for dischatge of catalytic scentilating compound according to the poresent invention, (21) represent compressor pumping air to discharge the bubles throug tht e pipe, (22) illustrate globulines, bubbles formed out of catalytic compound according to the presebt ivanntion, (22a) dripping iirigation type pipe used for sendiong bubbles into the air, or proximitt, (23) antenna for tracking th4 dimansion of the cata;ytic suspension thus disperes from the airplane (9).(24) illustrate the building on top which the antenna is stationed, (25) reprsent beam for environm ental snsing, and data aquasition, (26) a-e illustrate beams for communication of ground thruth monitoring statrion for global positioning oriantation shawn here for clarity between the helicopter, and the sattalite, and ground sensing equipment, (27) illustrate pulsed laser beam in the UVA,UVB,UVC, wherein it is used in the contectr Oooj the present ojvebtion to trigger the catalytic compounf accrdon top the preseb tnvwebtion, (29-30) illustrate both:diagnostic CW type beam for sensing, and PW type beam for triggering, (31) illustrate building for clarity and perspective ights, (32) illustrate ground level (earth) shawn her foe clarity extending right to the panoramic view of the schematic doiagram pf the oOressadsv tknvebntion., (33) illusdtrate a globulin, or buble weherein shawn herewith as bursting due to the acoustioc transpebnt thus generated accoprding to thep ursdb tojvebtion, (33a) post supe hydrophelicity effect shawn her for clarirty illustrating competitivce afvantegs of thr reaen onvention, wherein the remains of the bubble, or globulin is shawn her to fall, and thus ensure further appropriate mixing with the Oxygen ion ther air (21% free available ogygen content from which to take vitaol electrond for the efficnecies, and ehnacment Accprding p the present onwvntion, (34a) illustrate same processes as depicted by 33 with the exception of illustrating different hight/altitude (Y,Z,X) illudstration te ability od thre—resebty onvebntion to cover larger fireld of actionb as may be needed in incominf enfectious events or toxuc compouynds.

The method according to the present inventin is useful for catalytically protecting the surface of medical instrumentations, and sensitive or externally exposed, wounded or teard or burned body surface, or effecting internal blood and bodily fluids without effecting external surface of body (non invasive) comprising:Compounding a predeteermined volume of ultra-pure-water-based multi component system containing at least one liquid or gas or polymer, or bonding agent, or -catalytic-compound, PH stebilizers suspended, or expanded to contain at least one semi conductor, or catalyticaly potent or photoreactive components, or TIO2 photocatlyst having a predetermined acustic coupling density, or resonance potential;Calibrating semi opeque optomechanical properties of said catalytic compound for predetermined absorption band, or action spectrum, density, or predetermined refractive index, or refractive profile throughout for coupling or converting light or sound to and from predetermined destination; Innitiating acoustic optronic, or electronic diagnostic procedure, or objective using at least one tool or instrument which needs to be disinfected or made safe, or made biocompatiblelly free of noxious or infectious predetermined specie component, in a predetermined multi-component enviornm ent or threatening antigen, or wherein incoming infecsious events may cause health concern as a result of surface penetration or transmision through of noxious species; Encapsulating said instrument or tool with photoreactive, or catalytically potent compound whiping, or spraying, aplying or delivering or removing acces catalytic compound pre, or post said tool or instrument,is being utilized for said diagnostic objective or medical engineering procedure; Inserting said medical instruments, or device, or catalytically coated or charged tool into a predetermined receptive interface or conduit or chamber type geometry having a predetermined volume, and managable baundaries optomechanically impermieble to radiation from therein outwardly in the wavelength range of concern; Activating at least one radiation unit or interface directly, or continiously, in recurrently, or cyclically, or non recurrentl mode involving at least one high intensity source of light, and wherein said light is aligned to the end of at least one optical fiber, or wave-guide, or photogenic band gap, or aerobic or liquid wave-guide, or integrated arm, and wherein from other side of optical fiber, or bundle of fibers, or wave guide, or photonic band gap, or liquid light guide, or any combination thetreof, or directly illuminating or irradiating within said interface, the surface of said coated instrument, or tool, or catalytic compound therein; Triggering predetermined portion of said catalytic compound thin lyer encapsulation left on the surface of said tool, or instrument, or device photo-catalytically, or radically, thus inactivating, purifying, dissociating, and sterilizing the surface of said devices, or instruments in preperation for their associated cyclic, recurent, and non recurrent medical or engineering procedure, or working cycle, decontaminating or sterilizing, or disinfecting the surface of said tools or instrumentation to be more managable, and innociously safe for use within a predetermined period of time or duty cycle.

The present invention further relates to a method for treatment of surfaces having complex curvature wherein additional substances may be added for electronic trapping so as to facilitate prolonging the lifetime of free radical species, OH, produced according to the methodology of the present invention, such substances may be selected from liquids, gasses, or solids, in order to trap electron-pair holes, thus increasing beneficial sustaining, and delaying of the hydroxyl radial species lifetimes, increasing the quantum yield of efficiency according to the methodology of the present invention, especially beneficial for catalytic, scintillated, pH stabilized, oxygen charged toothpaste, or spray for the reduction of humic acids, and other factors effecting bad smells, tastes, substantially improving catalytic efficiencies of the methodology of the present invention.

1. Method for the advanced oxidation of dangerous chemical and biological sources suspected in particular regions, comprising the steps of;
(a) spraying over said regions a cloud of gas, vapors, microdroplets, droplets, or bubbles formed from at least one liquid solution containing at least one type of photocatalitic oxidizing substance;
(b) directing across said cloud at least one high intensity beam of light having wavelength of between 220 and 390 nanometer for triggering said cloud thereby causing a catalyzed activation that releases free radicals of said oxidizing substance in order to react with said chemical or biological sources.

2. A method for protection of public metropolitan areas, air volumes, surfaces, or combinations thereof using advanced oxidation processes triggered by pulsed UV laser, and photo-catalytic bubbles, droplets, jets, or combinations thereof, wherein said photo-catalytic components include at least one oxidizing agent, fluorescent, or phosphorous, or glowing, or reflecting, shifting, or vibrating or combination element such that once said photo reactive, or photo-catalytic compounds interact with light, it could be traceable for monitoring, and verification of bio-dosimetric dose, and chemical dissociation dosimetric values required for adequate inactivation and reduction, or minimization of threatening eventualities or contamination, cross contamination in any predetermined liquids, gases, surfaces, and combination.

3. Method for the advanced oxidation of dangerous chemical and biological sources suspected in particular regions according to any of the previous claims, further comprising scanning the cloud with the beam of light.

4. Method for the advanced oxidation of dangerous chemical and biological sources suspected in particular regions, according to any of the previous claims, wherein the beam of light is emitted from at least one laser unit.

5. Method for the advanced oxidation of dangerous chemical and biological sources suspected in particular regions, according to any of the previous claims, wherein the beam of light is emitted from at least one Laser unit selected from; solid state lasers, electrical discharge lasers, plasma driven lasers, semiconductor lasers, organic lasers, electron beam pumped lasers, free electron lasers, fiber lasers doped, or SASE/EA/FEL lasers, fiber lasers, diode pumped lasers, crystal based lasers, doped glass based lasers, FELs lasers, polymer lasers, PW/CW type lasers, quantum dote lasers, laser arrays, flush lamp pumped lasers, water lasers, photonic bandgap lasers, seeded or amplified lasers, time compressed or expanded lasers, q-switch type lasers, interactive harmonic lasers, acoustic-optical lasers, ultrasonic lasers, X-ray pumped lasers, Y pumped lasers, E-beam lasers, catalytic lasers, photoelectrocatalytic lasers, air lasers, ground stationary lasers, mobile and sub-miniaturized lasers, thin film type lasers, vapor lasers, water lasers, or photonic bandgapped lasers.

6. Method for the advanced oxidation of dangerous chemical and biological sources suspected in particular regions, according to any of the previous claims, wherein the beam of light is emitted from at least one laser unit, and is being pulsed.

7. Method for the advanced oxidation of dangerous chemical and biological sources suspected in particular regions, according to any of the previous claims, wherein the beam of light is emitted from at least one laser unit, and being pulsed in duty cycle of between 0.1 and 50 percent, in pulse repetition rate of between 1 Hz and 1 THz Gigahertz.

8. Method for the advanced oxidation of dangerous chemical and biological sources suspected in particular regions, according to any of the previous claims, wherein the beam of light is emitted from at least one laser unit, and is pulsed in short pulse duration having acoustic attack transient properties.

9. Method for the advanced oxidation of dangerous chemical and biological sources suspected in particular regions, according to any of the previous claims, wherein the cloud contains bubbles of size useful for opto-acoustic interaction between a beam of light scanning the cloud and the acoustic vibrations of the bubble mantles caused by the hit of the beam, which in turn, resulting in a multidirectional scattering of the beam, reflecting it to a plurality of surrounding bubbles or droplets.

10. Method for the advanced oxidation of dangerous chemical and biological sources suspected in particular regions according to any of the previous claims, wherein the solution contains phosphoric or fluorescense substance reactant to the light beam, to the light beam reflections from surrounding bubbles or droplets, or to particles released during the catalyzed activation, and wherein said method further comprising the step of tracing the light glowing from said phosphoric or fluorescense substance in order to identify whether or what portions of said region have been covered with an activated oxidizing substance.

11. Method for the advanced oxidation of dangerous chemical and biological sources suspected in particular regions according to any of the previous claims, further comprising the step of automatically deflecting the beam of light across the cloud according to light reflection data acquired from the cloud during the catalyzed activation of the oxidizing substance.

12. Method for the advanced oxidation of dangerous chemical and biological sources suspected in particular regions according to any of the previous claims, wherein the photo-catalytic bubbles or droplets contain at least one component of fluorescent, or phosphorous type such that the spectroscopic data acquisition thus accumulated provide for dosimetric value for inactivation of noxious species of biological or chemical origin, and thus the spectral distribution over a predetermined space, over a predetermined period of time is calibrated against specie specific calibration standards so as to identify the relevant thresholds needed for protecting, and treating application in predetermined surface area, volume or combination thereof.

13. Method for the advanced oxidation of dangerous chemical and biological sources suspected in particular regions according to any of the previous claims, further comprising measuring and calibrating the acoustic energy that light creates, and the light that acoustic energy creates. in order for establishing bio-dosimetric values or adapting the energy dose required for mineralizing or oxidizing predetermined toxic chemical species or biological noxious species,

14. Method for the advanced oxidation of dangerous chemical and biological sources suspected in particular regions according to any of the previous claims, wherein the solution is produced in real time or in immediate proximity to a disinfection procedure that is being implemented in an infection suspected site, and said solution is being prepared directly into flowing water supplied to sprinklers or bubble generators that create the cloud, wherein oxygen or any other essential dissolvent materials, are being dissolved into said flowing water during their flow to the sprinklers or bubble generators.

15. Method for the advanced oxidation of dangerous chemical and biological sources suspected in particular regions according to any of the previous claims, wherein the solution is produced in real time or in immediate proximity to a disinfection procedure that is being implemented in an infection suspected site, and said solution is being prepared directly into flowing water supplied to sprinklers or bubble generators that creates the cloud, wherein oxygen or any other essential dissolvent materials, are being dissolved into said flowing water during their flow to the sprinklers or bubble generators, and wherein the concentration of at least one of the solution ingredients is controlled by a computer and is case sensitive to the seriousness of dangerous chemical and biological sources under treatment.

16. Use of the method for the advanced oxidation of dangerous chemical and biological sources suspected in particular regions according to any of the previous claims, in outdoor regions; domestic regions; sea, air or ground vehicles; or space ships.

17. Use of the method for the advanced oxidation of dangerous chemical and biological sources suspected in particular regions according to any of the previous claims, for the disinfection of medical or surgical equipment and spaces.

18. Use of the method for the advanced oxidation of dangerous chemical and biological sources suspected in particular regions according to any of the previous claims, for the disinfection of toilet rooms.

19. Use of the method for the advanced oxidation of dangerous chemical and biological sources suspected in particular regions according to any of the previous claims, for catalytically protecting the surface of medical instrumentations, and sensitive or externally exposed, wounded or torn or burned body surface, or effecting internal blood and bodily fluids non invasively and without effecting external surface of body comprising Compounding a predetermined volume of ultra-pure-water-based multi component system containing at least one liquid or gas or polymer, or bonding agent, or -catalytic-compound, pH stabilizers suspended, or expanded to contain at least one semi conductor, or catalytically potent or photoreactive components, or TIO2 photocatalyst having a predetermined acoustic coupling density, or resonance potential; Calibrating semi opaque optomechanical properties of said catalytic compound for predetermined absorption band, or action spectrum, density, or predetermined refractive index, or refractive profile throughout for coupling or converting light or sound to and from predetermined destination; Innitiating acoustic optronic, or electronic diagnostic procedure, or objective using at least one tool or instrument which needs to be disinfected or made safe, or made biocompatibility free of noxious or infectious predetermined specie component, in a predetermined multi-component environment or threatening antigen, or wherein incoming infectious events may cause health concern as a result of surface penetration or transmission through of noxious species; Encapsulating said instrument or tool with photoreactive, or catalytically potent compound whiping, or spraying, applying or delivering or removing access catalytic compound pre, or post said tool or instrument, is being utilized for said diagnostic objective or medical engineering procedure; Inserting said medical instruments, or device, or catalytically coated or charged tool into a predetermined receptive interface or conduit or chamber type geometry having a predetermined volume, and manageable boundaries optomechanically impermeable to radiation from therein outwardly in the wavelength range of concern; Activating at least one radiation unit or interface directly, or continuously, in recurrently, or cyclically, or non recurrent mode involving at least one high intensity source of light, and wherein said light is aligned to the end of at least one optical fiber, or wave-guide, or photogenic band gap, or aerobic or liquid wave-guide, or integrated arm, and wherein from other side of optical fiber, or bundle of fibers, or wave guide, or photonic band gap, or liquid light guide, or any combination thereof, or directly illuminating or irradiating within said interface, the surface of said coated instrument, or tool, or catalytic compound therein; Triggering predetermined portion of said catalytic compound thin layer encapsulation left on the surface of said tool, or instrument, or device photo-catalytically, or radically, thus inactivating, purifying, dissociating, and sterilizing the surface of said devices, or instruments in preparation for their associated cyclic, recurrent, and non recurrent medical or engineering procedure, or working cycle, decontaminating or sterilizing, or disinfecting the surface of said tools or instrumentation to be more manageable, and innocuously safe for use within a predetermined period of time or duty cycle.

20. System for advanced oxidation of dangerous chemical and biological sources suspected in particular regions, comprising;
(a) means for the formation and distribution of a cloud of gas, vapors, microdroplets, droplets, or bubbles formed from at least one liquid solution containing at least one type of photocatalitic oxidizing substance, said means having at least one outlet for releasing or spraying said cloud;
(b) at least one laser unit generating light beam having properties that are useful for triggering said cloud, thereby causing a catalyzed activation that releases free radicals of said oxidizing substance in order to react with said chemical or biological sources.

21. System for advanced oxidation of dangerous chemical and biological sources suspected in particular regions, according to any of the previous claims, further comprising means for deflecting said light beam for scanning the cloud.

22. System for advanced oxidation of dangerous chemical and biological sources suspected in particular regions, according to any of the previous claims, further comprising means for deflecting said light beam for scanning the cloud, camera or sensor means for aquiring and for processing data which relates to light glowing from different portions of the cloud during its triggering or during its catalyzed activation, and means for steering the scanning automatically according to said data.

23. Bubble generator for forming a cloud of bubbles for the advanced oxidation of dangerous chemical and biological sources suspected in particular regions, comprising; at least one liquid container adapted to receiving a solution containing at least one type of photocatalitic oxidizing substance, means for gas compression, and at least one nozzle in liquid communication with the liquid reservoir, and in gas communication with the gas compression means.

24. Bubble generator for forming a cloud of bubbles for the advanced oxidation of dangerous chemical and biological sources suspected in particular regions, according to any of the previous claims, further comprising means for dissolving pure oxygen or any other essential dissolvent materials, into water flowing to the nozzle, for real time accomplishment of the solution.

25. Use of the system for the advanced oxidation of dangerous chemical and biological sources suspected in particular regions according to any of the previous claims, in outdoor regions; domestic regions; sea, air or ground vehicles; or space ships.

26. Use of the method for the advanced oxidation of dangerous chemical and biological sources suspected in particular regions, according to any of the previous claims, for catalytically protecting the surface of medical instrumentations, and sensitive or externally exposed, wounded or teard or burned body surface, comprising: Compounding a multi component system containing at least one liquid or gas or carbomer, or bonding agent forming a micro, and macro mollecular hybridization network having large external surface area within an ultra-pure water base pre-earated, or pre-charged with oxygen, or diatomic oxygen, of peroxide, or H2O2, or semi conductor photocatalytic particles mixed to form variable densities, multi resonance catalytic coupling compound featuring polytextural fromation from about heavy gel to about light spray, from about 1 m3 to about sevarl microdroplets; Calibrating semi opeque optomechanical properties of said catalytic compound for predetermined absorption band, or action spectrum, for coupling or amplifying, diagnosing or monitoring, observing life forms converting light or ultra-sound to and from predetermined origin of vibrations in line with available vibrational excitation stage required to break the bond by light between at least one components within said multicomponent photocatalytic photoreactive system; Innitiating acoustic optronic, or electronic diagnostic procedure, or objective using at least one tool or instrument which needs to be disinfected or made safe, or made biocompatiblelly free of noxious or infectious predetermined specie components; Encapsulating said instrument or tool with photoreactive, or catalytically potent compound whiping, or spraying, aplying or delivering or removing acces catalytic compound pre, or post medical procedure, living thin film of catalytic compound on said tool, or instruments, or device; lnserting at least one medical instrument, or device, catalytically and temporerily coated or charged encapsulated tool into at least one receptive interface being a conduit or chamber type geometry; Activating at least one radiation unit or interface directly, or continiously, in recurrently, or cyclically, or in non recurrentl mode involving at least one high intensity source of light, and wherein said light is aligned to the end of at least one optical fiber, or wave-guide, or photogenic band gap, or aerobic or liquid wave-guide, or integrated arm, and wherein from other side is position or suported or held at proximity to the surface of said coated instrument, or tool, or catalytic compound therein; Triggering predetermined portion of said catalytic compound thin lyer encapsulation left, cover, or sprayed or applied on to the the surface of said tool, or instrument, or device, or body cut sore, or wound, or burn, thus ensuring safety photo-catalytically, or radically, thus inactivating, purifying, dissociating, and sterilizing the surface of said devices, or instruments in preperation for their associated cyclic, recurent, and non recurrent medical or engineering procedure over apredetermined period of time.

27. Method for catalytic surface treatment according to any of the previous claims, comprising; non invasive, non contact, non interfering treatment methodology, wherein, by remote, inociously biocompatible automatically triggering through coupling predetermined aportioned predetermined light of expanded ultra pure water based electro-photocatalytic coupling compound, and all acousto-optronic accurate triggerring devices for disinfection and sterilization ensuring safe surface treatment of medical instrumentation, and external sensative tissue damage events, throughout associated areas of body thereof in real and step time process initiation format Compounding with water Calibrating with sound or light Innitiating with light intensities Encapsulating in catalytic thin layer Inserting coated Activating therein Triggering by light for inactivation over predetermined period of time.

28. A device comprising: at least one laser, or high intensity pulsed, or continuous, or hybrid combination of light sources is operating at pulse repetition frequencies from about 1 Hz, to about 1 THz.

29. A device comprising: at least one laser, or high intensity light source operatively utilized at pulse repetition frequencies from about 1 Hz, to about 1 THz.

30. A device comprising: at least one laser, or high intensity light source is projecting its beam of light directly, or through waveguides to the surface of the instrument, tools, or wounds, or burns.

31. A device for catalytic surface treatment using the method of the present invention in line with any of the preceeding claims, comprising: At least one receptive interface or support means or coated particle from PET, or polyolefin, or polyamide, or polycarbonate, or polyesteramide, or polyester or any resine combination thereof having refractive index profile for partial, or total internal reflection therein, or throughout, or coated oe combined with TIO2 semi conductor photocatalysts for equalizing the concentration of bacteria, viruses, cysts, phatogens, or biological, or organic, non organic, or toxic or noxious species therein, around, externally or combination when exposed to high peak power high intensity pulsed sub microsecond UVA UVB UVC laser light.

32. A device for disinfection of packaging of medical instrumentation and tools using the method of the present invention according to any of the preceding claims comprising; at least one beam homogenizer, or micro positioning stage, or optical lense, or array of lenses, or colimator optics, expansion optics, focusing or defocusing optics, difractive, or non fifractive optics, mirrors, prizma optical fibers, photonic waveguides, bandgap waveguides, liquid wave guides, aerobic non toxic waveguides, diagnostic semi conductor coated surfaces, or any combination thereof is used to shape the beams of light utilized for the purpose of reducing the population, or concentration of noxious species present in the predetermined volume of liquids, or gases in the packaging without damaging the packaging, and without causing migration of molecules from the packaging to the liquid or gas therein while in suspended, suspension or multi or single component system frame work.

33. A device using the method of the present invention and all of its preceeding claims, comprising: the prteffered mode for the packaging being penetrated is made out of PET, or polyolefin, or polyamide, or polycarbonate, or polyesteramide, or polyester or any resine combination thereof having refractive index profile for partial, or total internal reflection therein for disinfection, or equlization of the concentration of contaminents, or noxious species therein, i.e. in the packaging containing the predetermined volume of liquids, or gas therein.

34. A device as claimed in any of the previous claims wherein at least one Nd:Yag laser is operating at THG, third harmonic generation as preffered mode for the production of at least one pulse of light having wavelength of 355 nm, and wherein each time duration of said pulses of light is from about 1s, to about 1 fs, and wherein repetition rates of said laser light source is from about 1 Hz to about 1 THz for the preffered mode of triggering catalytic reaction, and or disinfection or sterilization of surface or dimension.

35. A device for quality control applications comprising a preffered mode especially beneficial for packaging materials having high refractive index selected from from Polyolefine, polyamide, polycarbonate, polyesteramide, polyester resine combination thereof, or terephtalate (PET), Polybutylen terephtalate (PBT), polyalkyl terephtalate, polyethylene naphtalate (PEN), polyalkyl naphtalate (PETG) having adequate refractive index profile therein for guiding light throughout. Especially beneficial for disinfection through the packaging by UVA laser, operating in pulsed mode, beneficial for quality control applications in mass production lines of mineral waters, flavored waters, beverages, juices, liquids, gasses, food based products, and medical preperation including the production, and polishing of insulin based products.

36. A device wherein the polymers selected from from Polyolefine, polyamide, polycarbonate, polyesteramide, polyester resine combination thereof, or terephtalate (PET), Polybutylen terephtalate (PBT), polyalkyl terephtalate, polyethylene naphtalate (PEN), polyalkyl naphtalate (PETG) having adequate refractive index profile therein for guiding light throughout, and wherein the light from a high intensity source of light is guided to reach the surface of the medical instrument, or tool, then activates photoreactive layer sorrounding said tool, or instruments forming fire wall barrier technology preventing external health threatening bacteria, viruses, and phathogenes from penetrating inside sensitive areas, or systems.

37. A device using the method of the present invention wherein according to any of the preceeding claims the cork or lid or bottom of the packaging, or any layer in the packaging is made out of from Polyolefine, polyamide, polycarbonate, polyesteramide, polyester resine combination thereof, or terephtalate (PET), Polybutylen terephtalate (PBT), polyalkyl terephtalate, polyethylene naphtalate (PEN), polyalkyl naphtalate (PETG), PET/PEN, PET/nylon, PET/Evoh, PET/Evoh copolymer or blend with ethylen-meth) acrylic acid, or combination with ultrapure water based catalytic compound having adequate refractive index profile therein for guiding light throughout. Especially beneficial for disinfection through the multi textural, multiple curvatures surface of medical instruments, tools, and engineering, and biotechnological devices having loaded working cycle.

38. A device using the method of the present invention and all of its preceeding claims, comprising: A method for surface treatment and for sterilization using modular, structurally yielding Oxygen Charged (SYOC) U.P.W, PH stebilized, carbomer variable photo-catalytic-water or air suspension, suspended polymeric particles expanded coated and calibrated to be mobilized by pulsed UVA/UVB/UVC laser light and all optronic delivery and triggering devices for dimensional use thereof

39. A device using the method of the present invention according to any of its preceeding claims wherein the the preffered mode for sterilizing dental accesories, tools and medical instrumentation, comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional use thereof.

40. A device using the method of the present invention according to any of its preceeding claims wherein the the preffered mode for sterilizing tools accesories, and instrumentation used in vetrinaric medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional use thereof.

41. A device using the method of the present invention according to any of its preceeding claims wherein the preffered mode for sterilizing tools accesories, and instrumentation used in emergency & field medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional use thereof.

42. A device using the method of the present invention according to any of its preceeding claims wherein the preffered mode for sterilizing tools accesories, and instrumentation used in medical engineering involved inmedical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional use thereof.

43. A device using the method of the present invention according to any of its preceeding claims wherein the preffered mode for sterilizing tools accesories, and instrumentation used in emergency & field medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional use thereof.

44. A device according to the method of the present invention according to any of its preceeding claims wherein the preffered mode for sterilizing tools accesories, and instrumentation used in small local autoclaving, or large central disinfection chambers for Surgical instrumentation of medical treatment procedures comprising: Triggering surface treatment and or sterilization using modular, structurally yielding Oxygen Charged (SYOCH) U.P.W, PH stabilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional use thereof.

45. A device using the method of the present invention according to any of its preceeding claims wherein the preffered mode for sterilizing tools accesories, and instrumentation used from Surgical instrumentation kits, autoclaving, emergency & field, medical engineering, vetrinaric, dental, wherein the preffered mode according to the specific applications preffered mode of operation will necesitate integraton or assimilation of the laser light source to be sellcted from (a) Gas discherge laser, (b) diod pumped lasers, (c) plasma discharged lasers, (d) solid state lasers, (e) semi conductor lasers, (f) crystal type of lasers, (g), X-rays pumped lasers, (h) E-beam pumped gas lasers types, FEL (Free Electron Laser amplifier), (j) EA/FEL (Electrostatically Accelerated Free Electron Laser), or organic laser types or any combination thereof for providing the necessary potons at the appropriate wavelength, tuning energy or energy density required for triggering and acoustooptical utilization.

46. A device using the method of the present invention according to any of its preceeding claims wherein the preffered mode for sterilizing tools accesories, and instrumentation used in cosmetics, plastic surgery from medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

47. A device using the method of the present invention according to any of its preceeding claims wherein the preffered mode for sterilizing tools accesories, and instrumentation used in alternative medicine or alternative medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

48. A device using the method of the present invention according to any of its preceeding claims wherein the preffered mode for sterilizing tools accesories, and instrumentation used in aquapuncture or aquapressure medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

49. A device using the method of the present invention according to any of its preceeding claims wherein the preffered mode for sterilizing tools accesories, and instrumentation used in gynicologycal medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

50. A device using the method of the present invention according to any of its preceeding claims wherein the preffered mode for sterilizing tools accesories, and instrumentation used in angioplastic medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

51. A device using the method of the present invention according to any of its preceeding claims wherein the preffered mode for sterilizing tools accesories, and instrumentation used in cardiac vascular medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

52. A device using the method of the present invention according to any of its preceeding claims wherein the preffered mode for sterilizing tools accesories, and instrumentation used in darmatological medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

53. A device using the method of the present invention according to any of its preceeding claims wherein the preffered mode for sterilizing tools accesories, and instrumentation used in PDT medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

54. A device using the method of the present invention according to any of its preceeding claims wherein the preffered mode for sterilizing tools accesories, and instrumentation used in peridental medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

55. A device using the method of the present invention according to any of its preceeding claims wherein the preffered mode for sterilizing tools accesories, and instrumentation used in cancer medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

56. A device using the method of the present invention according to any of its preceeding claims wherein the preffered mode for sterilizing tools accesories, and instrumentation used in allergenic medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

57. A device using the method of the present invention according to any of its preceeding claims wherein the preffered mode for sterilizing tools accesories, and instrumentation used in geriatric medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

58. A device using the method of the present invention according to any of its preceeding claims wherein the preffered mode for sterilizing tools accesories, and instrumentation used in pediatric medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

59. A device using the method of the present invention according to any of its preceeding claims wherein the preffered mode for sterilizing tools accesories, and instrumentation used in general operation theater medical treatment procedures comprising; Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

60. A device using the method of the present invention according to any of its preceeding claims wherein the preffered mode for sterilizing tools accesories, and instrumentation used in treating burns, and cuts, sores, and pressure wounds eventualities in medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

61. A device using the method of the present invention according to any of its preceeding claims wherein the preffered mode for sterilizing tools accesories, and instrumentation used in transplant medical procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

62. A device using the method of the present invention according to all of its preceding claims wherein the preferred mode for sterilizing tools accessories, and instrumentation used in dialysis medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

63. A device using the method of the present invention according to any of its preceeding claims wherein the preffered mode for sterilizing tools accesories, and instrumentation used in eye surgery or medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

64. A device using the method of the present invention according to any of its preceeding claims wherein the preffered mode for sterilizing tools accesories, and instrumentation used in administrating anathstetics during plurality of medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

65. A device using the method of the present invention according to all of its preceding claims wherein the preferred mode for sterilizing tools accessories, and instrumentation used in physiotherapy treatment procedures comprising: Triggering surface treatment and or sterilization using modular, structurally yielding Oxygen Charged (SYOCH) U.P.W, PH stabilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to manageable levels within a predetermined space over a predetermined period of time, and wherein Activating on all peak power optronic delivery and triggering apparatuses synched for dimensional acoustooptical utilization thereof.

66. A device using the method of the present invention according to all of its preceding claims wherein the preferred mode for sterilizing tools accessories, and instrumentation used in toxicology or decontamination of medical treatment procedures areas comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

67. A device using the method of the present invention according to any of its preceeding claims wherein the preffered mode for sterilizing tools accesories, and instrumentation used in nose ear, and throat doctors administrating medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

68. A device using the method of the present invention according to any of its preceeding claims wherein the preffered mode for sterilizing tools accesories, and instrumentation used in brain surgery medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

69. A device using the method of the present invention according to any of its preceeding claims wherein the preffered mode for sterilizing tools accesories, and instrumentation used in ultrasound medical treatment and diagnostics procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

70. A device using the method of the present invention according to any of its preceeding claims wherein the preffered mode for sterilizing tools accesories, and instrumentation used in endoscopic medical diagnostic treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

71. A device using the method of the present invention according to any of its preceeding claims wherein the preffered mode for sterilizing tools accesories, and instrumentation used in stenting or angioplastic medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

72. A device using the method of the present invention according to any of its preceeding claims wherein the preffered mode for sterilizing tools accesories, and instrumentation used in first aid medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

73. A device using the method of the present invention according to any of its preceeding claims wherein the preffered mode for sterilizing tools accesories, and instrumentation used in respiratory medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

74. A device using the method of the present invention according to any of its preceeding claims wherein the preffered mode for sterilizing tools accesories, and instrumentation used in medical treatment procedures for food poisening comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

75. A device using the method of the present invention according to any of its preceeding claims wherein the preffered mode for sterilizing tools accesories, and instrumentation used in blood transfusion and processing during medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

76. A device using the method of the present invention according to any of its preceeding claims wherein the preffered mode for sterilizing tools accesories, and instrumentation used in manicure, and pedicure cosmetic treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

77. A device using the method of the present invention according to any of its preceeding claims wherein the preffered mode for sterilizing tools accesories, and instrumentation used in cleaning ships, plains, or vehicles treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

78. A device using the method of the present invention according to any of its preceeding claims wherein the preffered mode for sterilizing tools accesories, and instrumentation used in cleaning traceable air polution comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles, or bubles comprising liquids, and gases expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

79. A device using the method of the present invention according to any of its preceeding claims wherein the preffered mode for sterilizing tools accesories, and instrumentation used in domestic cleaning, in locations, such as sanitary support means, sinks, ovens, fridges, microwave ovens, heaters, coolers, support means, copboards, tiles, floor, ceilings, medical treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

80. A device using the method of the present invention according to any of its preceeding claims wherein the preffered mode for sterilizing tools accesories, and instrumentation used in cleaning rubbish dumps comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

81. A device using the method of the present invention according to any of its preceeding claims wherein the preffered mode for sterilizing tools accesories, and instrumentation used in cleaning chips in electronic industries treatment procedures comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

82. A device using the method of the present invention according to any of its preceeding claims wherein the preffered mode for sterilizing tools accesories, and instrumentation used in optical washing machines applying catalytic treatment procedures to fabrics comprising: Triggering surface treatment and or sterilization using modular, structurally yielding Oxygen Charged (SYOCH) U.P.W, PH stebilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

83. A device using the method of the present invention according to any of its preceeding claims wherein the preffered mode for sterilizing tools accesories, and instrumentation used in food and beverage treatment production, and packaging procedures on sites comprising: Triggering surface treatment and or sterilization using modular, structurally yielding Oxygen Charged (SYOCH) U.P.W, PH stabilized, carbomer variable photo-catalytic-water or air suspension, suspended polymeric plurality particles expanded coated and calibrated to be mobilized by pulsed UVA/UVB/UVC laser light reducing the level of toxic noxious species below concentration, CFU, or danger threshold to managable levels within a predetermined space over a predetrmined period of time, and wherein Activating on all peak power optronic delivery and triggering aperatuses synched for dimensional acoustooptical utilization thereof.

84. A device using the methodology of the present invention for effecting internal blood and bodily fluids without effecting external surface of body non invasive, according to any of the preceding claims comprising:Calibrating semi opeque optomechanical properties of skin, and/or said catalytic compound of the present invention for predetermined absorption band, or action spectrum, density, or predetermined refractive index, or refractive profile throughout for coupling or converting light or sound to and from predetermined destination, and for enhancing the coupling efficiency, and absorption spectrum of the blood flow therein; Innitiating acoustic optronic, or electronic diagnostic procedure, or objective using at least one tool or instrument; Activating at least one radiation unit or interface directly, or continiously, in recurrently, or cyclically, or non recurrentl mode involving at least one high intensity source of light, or pulsed Vis, UVA, UVB laser and wherein light from the laser is aligned to the end of at least one optical fiber, or wave-guide, or photogenic band gap, or aerobic or liquid wave-guide, or tapered guide or integrated arm, and wherein from other side of optical fiber, or bundle of fibers, or wave guide, or photonic band gap, or liquid light guide, or any combination thetreof delivering light to/from external surface of body, or directly illuminating or irradiating within said interface, the surface of the outer body in a predetermined energy density, cumulative dose response curve, over a predetermined surface area, over a predetermined period of time or duty cycle.

85. A device using the methodology of the present invention for effecting internal blood and bodily fluids without effecting external surface of body (non invasive), according to any of the preceding claims comprising: Compounding a predeteermined volume of ultra-pure-water-based multi component system containing at least one liquid or gas or carbomer, or bonding agent, or -catalytic-compound, PH stebilizers suspended, or expanded to contain at least one semi conductor, or catalyticaly potent or photoreactive components, or TIO2 photocatlyst, or chemical precursor, or chamical biocompatible marker or colorimetric component having a predetermined acustic coupling density, or resonance potential; Calibrating semi opeque optomechanical properties of said catalytic compound for predetermined absorption band, or action spectrum, density, or predetermined refractive index, or refractive profile throughout for coupling or converting light or sound to and from predetermined destination, and for enhancing the coupling efficiency, and absorption spectrum of the blood flow therein; Innitiating acoustic optronic, or electronic diagnostic procedure, or objective using at least one tool or instrument which needs to be disinfected or made safe, or made biocompatiblelly free of noxious or infectious predetermined specie component, in a predetermined multi-component enviornm ent or threatening antigen, or wherein incoming infecsious events may cause health concern as a result of surface penetration or transmision through of noxious species, or wherein blood flow therein body contain noxious species concentration above threshold for safety risking health; delivering seperately photoreactive, or catalytically potent compound whiping, or spraying, aplying or delivering, infusing inserting, injecting, or activating intake of catalytic compound pre, or post exposure to pulsed light of UVA, laser or medical engineering procedure; Inserting or attaching said medical instruments, or device, or optically charged delivery tool into a predetermined receptive interface or conduit or chamber type geometry on the outer surface of the body having a predetermined volume, and managable baundaries optomechanically permieble to wavelength from about 350 nm to about 999 nm in the effective wavelength range approproate; Activating at least one radiation unit or interface directly, or continiously, in recurrently, or cyclically, or non recurrentl mode involving at least one high intensity source of light, or pulsed Vis, UVA, UVB laser and wherein light from the laser is aligned to the end of at least one optical fiber, or wave-guide, or photogenic band gap, or aerobic or liquid wave-guide, OR tapered guide or integrated arm, and wherein from other side of optical fiber, or bundle of fibers, or wave guide, or photonic band gap, or liquid light guide, or any combination thetreof delivering light to/from external surface of body, or directly illuminating or irradiating within said interface, the surface of the outer body in a predetermined energy density, cumulative dose response curve, over a predetermined surface area, over a predetermined period of time or duty cycle.

86. A device using the methodology of the present invention for effecting internal blood and bodily fluids without effecting external surface of body (non invasive), according to any of the preceding claims comprising: attaching optical waveguides, and diffusing elements to predetermined location externally to the human or animal body; Activating at least one radiation unit having high intensity source of monochromatic light at about 355 nm to about 999 nm, at energy densities from 0.001 mW/Cm2, to about 200 mW/Cm2; exposing synchroniousylly different part of the external relatively exposed blood vessels, such as found in areas sellected from head, legs, hands, wrists, feets, neck, forhead, stomech, belly, rectum, or any combination thereof to energy from said light source over a predetermined surface area, over a predetermined period of time thus reducing the population of noxious species in the blood therein without damaging external, or internal blood or physiological components.

87. A device using the methodology of the present invention for effecting internal blood and bodily fluids without effecting external surface of body (non invasive), according to any of the preceding claims comprising: attaching optical waveguides, and diffusing elements to predetermined location externally to the human or animal body; Activating at least one radiation unit having high intensity source of monochromatic light at about 355 nm to about 999 nm, at energy densities from 0.001 mW/Cm2, to about 200 mW/Cm2; exposing synchroniousylly different part of the external relatively exposed blood vessels, such as found in areas sellected from head, legs, hands, wrists, feets, neck, forhead, stomech, belly, rectum, or any combination thereof to energy from said light source over a predetermined surface area, over a predetermined period of time thus reducing the population of noxious species in the blood therein without damaging external, or internal blood or physiological components thus facilitating non invasive disinfection of intenally flowing blood components.

88. A method and device using the methodology of the present invention for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the duty cycle of procedures thus performed in these respective field are shortened, and time and resources saved, effectively reducing the population of noxious species, thus increasing the level of health, and therapeutic applications of devices according to the methodology of the present invention.

89. A method and device using the methodology of the present invention for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the duty cycle of procedures thus performed in these respective field when using multicomponent catalytic U.PW based compound or coupling gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

90. A method and device using the methodology of the present invention for sterilizing and disinfecting the surface of medical instrumentation, according to any of the preceding claims, comprising te light which provide the triggering signal is produced by flush lamps, UVA, UVB, UVC laser pumped by diodes bar arrays, flush lamp pumped Nd; Yag lasers, sub microsecond lasers, gas discharge lasers, hybrid of CW/PW integration, X-ray pumped lasers, E-beam pumped lasers, FEL, AEFEL lasers, and semi conductor lasers, or space charged excitation charged lasers or any combination thereof for the delivery of static, stable, or cumulative dose made out of a plurality of micro, or macro pulses from 1 photon per second to about 100 Billion Photons per second, at repetition rates from about 1 Hz, to about 10 THz, at wavelength from about 355 nm to about 999 nm, and wherein acousto-optical-interaction are thus been facilitated as result of translational relationships between acoustics, electronic, and optics causing resonance useful to determined optical masking od certain mollecules while making other mollecules in proximity more suseptible for predetermined specie specific action spectrum, thus inactivating specie specific calibration standards using the acuosto-optronic interaction using the methodology of the present invention.

91. A method and device using the methodology of the present invention for sterilizing and disinfecting the surface of medical instrumentation, according to any of the preceding claims, comprising the beam is delivered directly on to the surface of the instrument or tool, or the beam is cooupled to the end of a waveguide interface or integrated arm for remote delivery of the appropriate energy levels to the destination, and wherein such bundle formation, harness, or multisplit level optical waveguide is adjusted as to illuminateor irradiate the surface area at the appropriate energy density levels require for implementing at least one biodosimetric curve against at least one noxious specie which causes health concern ahen found in high population concentration.

92. A method and device using the methodology of the present invention for sterilizing and disinfecting the surface of medical instrumentation, according to any of the preceding claims, comprising: Preferred mode of utilization for emergency medicine requiring fast responses to incoming or inflicted infectious events wherein the duty cycle of procedures thus performed in these respective field when using multicomponent catalytic U.PW based compound or coupling gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention facilitating safe sterilized surface area on tools, or body external surface.

93. A method and device for sterilizing and disinfecting the surface of medical instrumentation, according to any of the preceding claims, comprising: Preferred mode of utilization for anesthesiology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

94. A method and device for sterilizing and disinfecting the surface of medical instrumentation, according to any of the preceding claims, comprising: Preferred mode of utilization for dermatology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

95. A method and device for sterilizing and disinfecting the surface of medical instrumentation, according to any of the preceding claims, comprising: Preferred mode of utilization for gastroenterology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

96. A method and device for sterilizing and disinfecting the surface of medical instrumentation, according to any of the preceding claims, comprising: Preferred mode of utilization for Ob-Gyn wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

97. A method and device for sterilizing and disinfecting the surface of medical instrumentation, according to any of the preceding claims, comprising: Preferred mode of utilization for oncology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

98. A method and device for sterilizing and disinfecting the surface of medical instrumentation, according to any of the preceding claims, comprising: Preferred mode of utilization for opthalmology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

99. A method and device for sterilizing and disinfecting the surface of medical instrumentation, according to any of the preceding claims, comprising: Preferred mode of utilization for ostheopathy wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

100. A method and device for sterilizing and disinfecting the surface of medical instrumentation, according to any of the preceding claims, comprising: Preferred mode of utilization for pain management wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

101. A method and device for sterilizing and disinfecting the surface of medical instrumentation, according to any of the preceding claims, comprising: Preferred mode of utilization for pathology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

102. A method and device for sterilizing and disinfecting the surface of medical instrumentation, according to any of the preceding claims, comprising: Preferred mode of utilization for pediatrics wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

103. A method and device for sterilizing and disinfecting the surface of medical instrumentation, according to any of the preceding claims, comprising: Preferred mode of utilization for podiatry wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

104. A method and device for sterilizing and disinfecting the surface of medical instrumentation, according to any of the preceding claims, comprising: Preferred mode of utilization for radiology wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

105. A method and device for sterilizing and disinfecting the surface of medical instrumentation, according to any of the preceding claims, comprising: Preferred mode of utilization for cardiothoracic surgery wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

106. A method and device for sterilizing and disinfecting the surface of medical instrumentation, according to any of the preceding claims, comprising: Preferred mode of utilization for cardiology and invasive diagnbostic and treatment procedures wherein the duty cycle of said procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

107. A method and device for sterilizing and disinfecting the surface of medical instrumentation, according to any of the preceding claims, comprising: Preferred mode of utilization for cosmetics, and plastic surgery wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

108. A method and device for sterilizing and disinfecting the surface of medical instrumentation, according to any of the preceding claims, comprising: Preferred mode of utilization for orthopedics wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

109. A method and device for sterilizing and disinfecting the surface of medical instrumentation, according to any of the preceding claims, comprising: Preferred mode of utilization for ENT wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

110. A method and device for sterilizing and disinfecting the surface of medical instrumentation, according to any of the preceding claims, comprising: Preferred mode of utilization for general surgery in wide diversified medical fields wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

111. A method and device for sterilizing and disinfecting the surface of medical instrumentation, according to any of the preceding claims, comprising: Preferred mode of utilization for endoscopic surgery/procedures wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

112. A method and device for sterilizing and disinfecting the surface of medical instrumentation, according to any of the preceding claims, comprising: Preferred mode of utilization for skin treatment, ear diorders, eay disorders, mouth disorders, throat disorders, dental disorders wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

113. A method for sterilizing and disinfecting the surface of medical instrumentation, according to any of the preceding claims, comprising: Preferred mode of utilization for external non invasive treatment by light at high energy density wherein the duty cycle of procedures thus performed in these respective field when using multicomponent catalytic U.PW based compound or directly coupling pulsecd UVA, UVB, UVC laser light directly, or in combination with/to gel, spray, or liquid or gas facilitating the shortening, and time and resources saving, as results of effectively reduction of the population of noxious species on surface or volume, within predetermined area or dimension, thus increasing the level of health, and therapeutic applications of devices according to the methodology of the present invention.

114. A method and device for sterilizing and disinfecting the surface of medical instrumentation, according to any of the preceding claims, comprising: Preferred mode of utilization for domestic cleaning and disinfection, and sterilization of aparatuses sellected from kitchens, sinks, bathrooms, infrastructural support means, floors, ceilings, air wherein the duty cycle of procedures thus performed in these respective field or domestic enviornm ent using multicomponent catalytic U.PW based compound or directly coupling pulsecd UVA, UVB, UVC laser light directly, or in combination with gel, spray, or liquid or gas facilitating the shortening, and time and resources saving, as results of effectively reduction of the population of noxious species on surface or volume, within predetermined area or dimension, thus increasing the level of health, and hygine applications of devices according to the methodology of the present invention.

115. A method and device for sterilizing and disinfecting the surface of medical instrumentation, according to any of the preceding claims, comprising: Preferred mode of utilization for domestic cleaning and disinfection, and sterilization of aparatuses sellected from kitchens, sinks, bathrooms, infrastructural support means, floors, ceilings, air enhancing super hydrophelicity wherein the duty cycle of procedures thus performed in these respective field or domestic enviornm ent using multicomponent catalytic U.PW based compound or directly coupling pulsecd UVA, UVB, UVC laser light directly, or in combination with gel, spray, or liquid or gas facilitating the shortening, and time and resources saving, as results of effectively reduction of the population of noxious species on surface or volume, within predetermined area or dimension, thus increasing the level of health, and hygine applications of devices according to the methodology of the present invention.

116. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the duty cycle of procedures thus performed in these respective field when using multicomponent catalytic U.PW based compound or coupling gel, spray, or liquid or gas, or tooth paste for catalytic, scintilating, repeatable triggering, thus facilitating the shortening, and time and resources saving, as results of effectively reduction of the population of noxious species such as pluck formation on surface or volume, within predetermined area or dimension, on teeth, or between teeth, ot gums, thus increasing the level of health, and therapeutic applications of devices according to the methodology of the present invention for preventative treatment as well as for damage limitation exercises, and for cleaning deep, and thoughroughlly the entire mouth.

117. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein acatalytic tooth paste is been used containing scintillating elementals causing efficient conversion from Visible, NIR, IR to UVA, UVB, UVC thus facilitating triggering, and re-triggering using light alone, in addition to using an optronic brush to apply said paste when 1st brushing teeth in the mornings, or cyclically, or non recurrently, cleaning the mouth continiousily throughout the day using light.

118. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the brush contain at least one optical fiber, difuser, aerobic, and non aerobic guide, support means, and power supply on board for triggering the catalytic action of catalytic paste repeatedly.

119. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the photocatalystic tooth paste is containing mint flavore or aromatic elements so as to esthetically fit behavoral pattern, and prefferances of producers, end users, and doctors, effectively increasing the cleanliness of he moutgh area.

120. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the brush used to triggered the photocatalytic tooth paste contain a sub-miniaturized radiation ubit having a relatively high intensity source of light from about 1 m/W Cm2, to about 180 mW Cm2, and wherein light from the brush is reaching to exposed said tooth paste throughout the mouth, teeth, and gums, and wherein after 1st application of the paste, repeatable triggering may occur just by exposing the thin film layer left on the tooth over several hours, so by introducing light to the mouth after brushing has occurs the catalytic processes according to the present invantioon continue to occur bringinh with them protection, and treatment benefits for the health of the mouth, and sorrounding area.

121. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the brush used to trigger the catalytic tooth paste is containing at least one LED, laser, flash lamp, quasy CW laser, hybrid, or integrated light source or guiding light from external light source in proxinitym or through the use of a photonic fiber, guide, or bundle of fibers.

122. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein only light is efectively treating the coated area after firsts application of the paste have occured.

123. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein catalytic tooth paste accrdong to ther present invention is reducing the humic acids, bad smells, and improving decay condition by reducing the concentration of noxious species, bacteria, viruses, cysts which may threat a mouth healthy condition.

124. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein global solar radiation is used to triggere the potocatalysts at about the 1 mW/Cm2, energy density, and wherein a scintillating elemental has been added, doped, spattered, vaporized, mixed to convert visible radiation to UVA, UVB, UVC from about 1 eV, to about 9.1 eV, and wherein condensing optics, and delivery interfaces guidethe light into the mouth area, surface, or exact location to be remedied.

125. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the catalytic scintillating toothe paste is stored, temporerily, or permanently in a tube, or capsule, conduit or chaimber prior to application, delivery, or triggering, or re-triggering, cyclically, recurrently, or non recurrently for the elimination of pluck, and noxious bacteria, and bad smell effect and humic acids.

126. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the catalytic scintillated compound according to TIO2 photocatalyst into homoginic distribution within the multi-component which characterizes the catalytic compound according to the present invention is stored, temporerily, or permanently in form of a light spray containing ph stebilized, water and oxygen charged carbomer based textural suspension and wherein scintillating elemental is used to inse.

127. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the catalytic scentilated compound according to the present invention is stored, temporerily, or permanently in a compressed form.

128. A method and device for sterilizing and disinfecting the surface of medical instrumentation, according to any of the preceding claims, comprising: Preferred mode of utilization for dentistry, general dentists, periodontists, prothodontists, pedodontists, pediatric dentists, endodontists, oral/maxillofacial surgeons, and orthodontists wherein the catalytic scentilated compound according to the present invention is stored, temporerily, or permanently in form of a light spray.

129. A method and device for sterilizing and disinfecting the surface of medical instrumentation, the mouth, and blood, non invasively according to any of the preceding claims, comprising: A preffered mode of geometrical utilization of devices using method of the present invention wherein the laser light source is sellcted from Gas discherge laser, diod pumped lasers, plasma discharged lasers, solid state lasers, semi conductor lasers, crystal type of lasers, X-rays pumped lasers, E-beam pumped gas lasers types, FEL (Free Electron Laser amplifier), EA/FEL (Electrostatically Accelerated Free Electron Laser), or organic laser types or any combination thereof.

130. A method and device for sterilizing and disinfecting the surface of medical instrumentation, the mouth, and blood, non invasively according to any of the preceding claims, comprising: A preffered mode of geometrical utilization of devices using method of the present invention wherein the laser light source is sellcted from Gas discherge laser, diod pumped lasers, plasma discharged lasers, solid state lasers, semi conductor lasers, crystal type of lasers, X-rays pumped lasers, E-beam pumped gas lasers types, FEL (Free Electron Laser amplifier), EA/FEL (Electrostatically Accelerated Free Electron Laser), or organic laser types or any combination thereof.

131. A method for disinfecting a target, comprising electrooptically exciting a water-based photoreactive compound with a high peak-power, high repitition rate, submicrosecond time domain, pulsed UV laser, wherein the water-based photoreactive compounds comprises a multi-component system having at least one pH stabilizer ot bonding agent, at least biocompatible polymer, a photocatalytic agent, and a predetermined oxygen charge.

132. A method for producing a photoreactive multi-component system comprising combining a photosensitive compound with at least one polymer, a pH stabilizer or bonding agent, ultrapure water, a photocatalytic agent, and dissolved oxygen.

133. A method according to claim 132, further comprising adding a scintilating agent having the ability to convert incoming light to an appropriate triggering wavelength.

134. A method according to claim 132, further comprising adding a component selected from the group consisting of: nutrients, food supplements, colors, flavors, medications, or carbomers, said carbomers for modifying the texture of said multi-component system.

135. A multi-component system made according to the method of any one of claims 132-134.

136. A device for disinfecting a target, comprising; at least one radiation unit having a high intensity source of light having a peak power energy density of between about 1*10-7 to 1*10-29 mJ/cm2 having a wavelength between 1 and 3000 nm and a repitition rate between 1 Hz to 180 THz; a nonaerobic, nontoxic optical waveguide in connection with said radiation unit for delivering radiation from said radiation unit to said target over a predetermined area and predetermined period of time.

137. A method for phototreatment of a target using the device of claim 112 comprising exposing said target area to radiation from said radiation unit for a predetermined period of time.

138. A method for treatment of surfaces having complex curvature wherein additional substances may be added for electronic trapping so as to facilitate prolonging the lifetime of free radical species, OH, produced according to the methodology of the present invention, such substances may be selected from `liquids, gasses, or solids, in order to trap electron-pair holes, thus increasing beneficial sustaining, and delaying of the hydroxyl radial species lifetimes, increasing the quantum yield of efficiency according to the methodology of the present invention, especially beneficial for catalytic, scintillated, pH stabilized, oxygen charged toothpaste, or spray for the reduction of humic acids, and other factors effecting bad smells, tastes, substantially improving catalytic efficiencies of the methodology of the present invention.
